SEQUENCE LISTING (13 sequences)

SEQ ID NO: 1 - protein, 14 aa, synthetic construct
  UNSURE 1..14: note = GLS antibody
KLDPRREGGDQRHS

SEQ ID NO: 2 - DNA, 21 bp, synthetic construct
  source 1..21: mol_type = other DNA
  misc_feature 1..21: note = SARS-CoV-2-spike-F
caactgaaatctatcaggccg

SEQ ID NO: 3 - DNA, 21 bp, synthetic construct
  source 1..21: mol_type = other DNA
  misc_feature 1..21: note = SARS-CoV-2-spike-R
accaacaccattagtgggttg

SEQ ID NO: 4 - DNA, 24 bp, synthetic construct
  source 1..24: mol_type = other DNA
  misc_feature 1..24: note = HCoV-OC43-F
cccaagcaaactgctacctctcag

SEQ ID NO: 5 - DNA, 24 bp, synthetic construct
  source 1..24: mol_type = other DNA
  misc_feature 1..24: note = HCoV-OC43-R
cccaagcaaactgctacctctcag

SEQ ID NO: 6 - DNA, 20 bp, synthetic construct
  source 1..20: mol_type = other DNA
  misc_feature 1..20: note = HCoV-229E-F
tctgccaagagtcttgctcg

SEQ ID NO: 7 - DNA, 20 bp, synthetic construct
  source 1..20: mol_type = other DNA
  misc_feature 1..20: note = HCoV-229E-R
tctgccaagagtcttgctcg

SEQ ID NO: 8 - DNA, 23 bp, synthetic construct
  source 1..23: mol_type = other DNA
  misc_feature 1..23: note = GLS-F
tgtcacgatcttgtttctctgtg

SEQ ID NO: 9 - DNA, 21 bp, synthetic construct
  source 1..21: mol_type = other DNA
  misc_feature 1..21: note = GLS-R
tcatagtccaatggtccaaag

SEQ ID NO: 10 - DNA, 21 bp, synthetic construct
  source 1..21: mol_type = other DNA
  misc_feature 1..21: note = GLS2-F
gcctgggtgatttgctctttt

SEQ ID NO: 11 - DNA, 22 bp, synthetic construct
  source 1..22: mol_type = other DNA
  misc_feature 1..22: note = GLS2-R
cctttagtgcagtggtgaactt

SEQ ID NO: 12 - DNA, 20 bp, synthetic construct
  source 1..20: mol_type = other DNA
  misc_feature 1..20: note = actin-F
catcgagcacggcatcgtca

SEQ ID NO: 13 - DNA, 21 bp, synthetic construct
  source 1..21: mol_type = other DNA
  misc_feature 1..21: note = actin-R
tagcacagcctggatagcaac

(19) United States
(12) Patent Application Publication
(10) Pub. No.: US 2025/0288577 A1
(43) Pub. Date: Sep. 18, 2025

(54) METHODS OF TREATING A VIRUS INFECTION AND METHODS OF INHIBITING VIRAL REPLICATION

(71) Applicant: CORNELL UNIVERSITY, Ithaca, NY (US)

(72) Inventors: Richard CERIONE, Ithaca, NY (US); Kai Su GREENE, Ithaca, NY (US); Gary WHITTAKER, Ithaca, NY (US)

(21) Appl. No.: 18/861,007

(22) PCT Filed: Apr. 28, 2023
(86) PCT No.: PCT/US2023/066392

§ 371 Date: Oct. 28, 2024

Related U.S. Application Data
(60) Provisional application No. 63/336,855, filed on Apr. 29, 2022.

(51) Int. Cl. A61K 31/473; A61K 31/426; A61K 31/433; A61K 31/454; A61K 31/4745; A61K 31/513; A61K 45/06; A61P 31/14

(52) U.S. Cl. CPC A61K 31/473; A61K 31/426; A61K 31/433; A61K 31/454; A61K 31/4745; A61K 31/513; A61K 45/06; A61P 31/14

(57) ABSTRACT

The present disclosure relates to a method of treating a virus infection in a subject, the method comprising administering to the subject a compound of either formula (I) or formula (II), or a pharmaceutically acceptable salt of formula (I) or formula (II). Also disclosed is a method of inhibiting viral replication, the method comprising contacting one or more cells infected with a virus with an effective amount of a compound of either formula (I) or formula (II), or a pharmaceutically acceptable salt of formula (I) or formula (II).

This application claims the priority benefit of U.S. Provisional Patent Application Ser. No. 63/336,855, filed Apr. 29, 2022, which is hereby incorporated by reference in its entirety.

This invention was made with government support under grant numbers GM122575 and CA201402 awarded by the National Institutes of Health. The government has certain rights in the invention.

### FIELD

The present disclosure relates to methods for treating a virus infection and methods of inhibiting viral replication.

### SEQUENCE LISTING

The instant application contains a Sequence Listing which has been submitted electronically in XML format and is hereby incorporated by reference in its entirety. Said XML copy, created on Apr. 28, 2023, is named 147402.009151.xml and is 17,228 bytes in size. No new matter is being introduced.

### BACKGROUND

Glutamine plays a key role in generating the building blocks necessary for the biosynthetic processes of various cells. Glutamine metabolism is often upregulated in aggressive cancer cells (Pavlova and Thompson, “Emerging Metabolic Hallmarks of Cancer,” Physiol Behav. 176(1):139-148 (2018); DeBerardinis et al., “The Biology of Cancer: Metabolic Reprogramming Fuels Cell Growth and Proliferation,” Cell Metab. 7(1):11-20 (2008); Altman et al., “From Krebs to Clinic: Glutamine Metabolism to Cancer Therapy,” Nat. Rev. Cancer 16(10):619-634 (2017); Vander Heiden et al, “Understanding the Warburg Effect: The Metabolic Requirements of Cell Proliferation,” Science 324(5930):1029-1033 (2009); De Berardinis and Chandel, “Fundamentals of Cancer Metabolism,” Sci. Adv. 2(5): e1600200 (2016); Vander Heiden and DeBerardinis, “Understanding the Intersections Between Metabolism and Cancer Biology.” Cell 168(4):657-669 (2017)). Normal healthy cells typically utilize glucose as their primary bioenergetic and biosynthetic material. However, cancer cells have been shown to reprogram their metabolism by increasing the utilization of glutamine as the primary nutrient to support their TCA cycle and to generate building blocks for biosynthetic activities necessary to support their high rates of proliferation and for their ability to survive cellular stresses (Pavlova and Thompson, “Emerging Metabolic Hallmarks of Cancer,” Physiol. Behav. 176(1):139-148 (2018); Finley L W S, “What is Cancer Metabolism?,” Cell 186(8):1670-1688 (2023); Wang et al., “Targeting Mitochondrial Glutaminase Activity Inhibits Oncogenic Transformation,” Cancer Cell 18(3):207-219 (2010); Dang et al., “MYC-Induced Cancer Cell Energy Metabolism and Therapeutic Opportunities,” Clin. Cancer Res. 15(21):6479-6483 (2009); Ferrer et al., “0-GlcNAcylation Regulates Cancer Metabolism and Survival Stress Signaling via Regulation of the HIF-1 Pathway,” Mol. Cell 54(5):820-831 (2014); Greene et al., “SIRT5 Stabilizes Mitochondrial Glutaminase and Supports Breast Cancer Tumorigenesis,” Proc. Natl. Acad. Sci. USA 116(52):26625-26632 (2019)). Members of the glutaminase family of enzymes catalyze the first step in glutamine metabolism, the hydrolysis of glutamine to glutamate, with glutamate then being converted to a-ketoglutarate by glutamate dehydrogenase to enter the TCA cycle.

Two genes, gls and gls2, encode the glutaminase enzymes in mammals (de la Rosa et al., “A novel Glutaminase Isoform in Mammalian Tissues,” Neurochemistry International 55:76-84 (2009)). Gls encodes KGA (kidney-type glutaminase) and the C-terminal truncated splice variant GAC (glutaminase C) (now collectively referred to as GLS) which are ubiquitously expressed in mammalian tissues, while gls2 encodes LGA (liver-type glutaminase, from here-on designated GLS2) and is primarily found in liver, pancreas, and brain (Altman et al., “From Krebs to Clinic: Glutamine Metabolism to Cancer Therapy,” Nat. Rev. Cancer 16(10):619-634 (2017); Ferrer et al., “O-GlcNAcylation Regulates Cancer Metabolism and Survival Stress Signaling via Regulation of the HIF-1 Pathway,” Mol. Cell 54(5):820-831 (2014); Katt et al., “A Tale of Two Glutaminases: Homologous Enzymes With Distinct Roles in Tumorigenesis,” Future Med. Chem. 9(2):223-243 (2017); and Campos-Sandoval et al., “Glutaminases in Brain: Multiple Isoforms for Many Purposes,” Neurochem. Int. 88:1-5 (2015)). GLS has been shown to be highly expressed in various types of cancers including basal-subtype triple negative breast cancer, glioblastoma, and pancreatic cancers (Wang et al., “Targeting Mitochondrial Glutaminase Activity Inhibits Oncogenic Transformation,” Cancer Cell 18(3):207-219 (2010) and Lukey et al., “Liver-Type Glutaminase GLS2 Is a Druggable Metabolic Node in Luminal-Subtype Breast Cancer,” Cell Rep. 29(1):76-88 (2019)), due to the actions of the transcription factors c-Myc and c-Jun (Gao et al., “C-Myc Suppression of miR-23a/b Enhances Mitochondrial Glutaminase Expression and Glutamine Metabolism,” Nature 458(7239):762-765 (2009) and Lukey et al., “The Oncogenic Transcription Factor c-Jun Regulates Glutaminase Expression and Sensitizes Cells to Glutaminase-targeted Therapy,” Nat. Commun. 7:1-14 (2016)). GLS2 was recently implicated in luminal-subtype breast cancer. It has been reported that allosteric inhibitors of GLS block cancer cell proliferation (Katt et al., “A Tale of Two Glutaminases: Homologous Enzymes With Distinct Roles in Tumorigenesis,” Future Med. Chem. 9(2):223-243 (2017) and Milano et al., “New Insights Into the Molecular Mechanisms of Glutaminase C Inhibitors in Cancer Cells Using Serial Room Temperature Crystallography,” J. Biol. Chem. 298(2):101535 (2022)), with CB839 being examined in a number of clinical trials as an anti-cancer drug (Campos-Sandoval et al., “Glutaminases in Brain: Multiple Isoforms for Many Purposes,” Neurochem. Int. 88:1-5 (2015) and Wicker et al., “Glutaminase Inhibition With Telaglenastat (CB-839) Improves Treatment Response in Combination With Ionizing Radiation in Head and Neck Squamous Cell Carcinoma Models,” Cancer Lett. 502:180-188 (2021)).

It has been well established that metabolic re-programming occurs during cancer progression, and similarly, there have been reports suggesting viruses may hijack the biosynthetic machinery of host cells and to be dependent upon glutamine metabolism for their replication and the generation of new viral particles. A traditional GLS small molecule inhibitor, Compound C, has been reported to inhibit Kaposi-sarcoma associated herpesvirus (KSHV) and Vaccinia virus (VACV), whereas its more potent derivative, CB839, was shown to block Adenovirus and influenza A (Mayer et al., “Hijacking the Supplies: Metabolism as a Novel Facet of Virus-host Interaction,” Front Immunol. 10(JULY):1-12 (2019); Thai et al., “MYC-induced Reprogramming of Glutamine Catabolism Supports Optimal Virus Replication,” Nat. Commun. 6(1):8873 (2015); and Fontaine et al., “Vaccinia Virus Requires Glutamine but Not Glucose for Efficient Replication,” J Virol. 88(8):4366-4374 (2014)). However, thus far it has not been shown whether glutamine metabolism is essential for Coronavirus infection and if GLS inhibitors might be effective in blocking their replication.

Coronaviruses are a group of enveloped viruses that contain a single positive RNA strain and “Corona”-like spike proteins extending from their envelopes. Seven types of Coronaviruses have been reported to infect humans. The Severe acute respiratory syndrome 2 (SARS-CoV-2) pandemic which began in 2019 has taken many lives across the globe, with multiple viral mutant strains having emerged since that time. Human Coronaviruses OC43 (HCoV-OV43) and 229E (HCoV-229E) are far less lethal and typically only give rise to common cold symptoms. Each Coronavirus family member has a slightly distinct spike protein and consequently binds to different receptors to enter cells. Thus, SARS-CoV2 binds to the Angiotensin converting enzyme 2 (ACE2) receptor (V'kovski et al., “Coronavirus Biology and Replication: Implications for SARS-CoV-2,” Nat. Rev. Microbiol. 19(3):155-170 (2021); Shapira et al., “A TMPRSS2 Inhibitor Acts as a pan-SARS-CoV-2 Prophylactic and Therapeutic,” Nature 605(7909):340-348 (2022); Zhou et al., “A Pneumonia Outbreak Associated With a New Coronavirus of Probable Bat Origin,” Nature 579(7798):270-273 (2020); and Beyerstedt et al., “COVID-19: Angiotensin-converting Enzyme 2 (ACE2) Expression and Tissue Susceptibility to SARS-CoV-2 Infection,” Eur. J. Clin. Microbiol. Infect. Dis. 40(5):905-919 (2021)) HCoV-OC43 engages the N-acetyl-9-O-acetyl neuraminic acid receptor, and HCoV229E interacts preferentially with human aminopeptidase (hANP)(Liu et al., “Human Coronavirus-229E, -OC43, -NL63, and -HKU1 (Coronaviridae),” In: Encyclopedia of Virology Elsevier 428-440 (2021); Sriwilaijaroen and Suzuki, “Host Receptors of Influenza Viruses and Coronaviruses-Molecular Mechanisms of Recognition,” Vaccines 8(4):1-47 (2020); Tang et al., “Human Coronaviruses: Origin, Host and Receptor,” J. Clin. Virol. 155:105246 (2022)). SARS-CoV2 and HCoV-OC43 are members of the Coronavirus beta sub-group family, while HCoV-229E is a member of the alpha sub-group. Since the onset of the pandemic, antibody and RNA vaccines targeting SARS-CoV2 continue to be developed. However, each has its limitations, thus there continues to be a pressing need to identify new therapeutic strategies that will provide broad protection against new virus strains and mutants which are likely to emerge.

The present disclosure is directed to overcoming these and other deficiencies in the art.

### SUMMARY

A first aspect of the disclosure relates to a method of treating a virus infection in a subject. The method comprises administering to the subject a compound of either:
(A) formula (I)

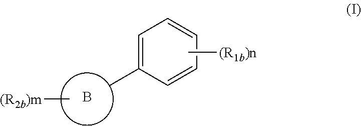

wherein:
m and n are integers from 1 to 4;
B is a substituted or unsubstituted mono or polycycle, wherein the monocycle is an aryl ring, a heteroaryl ring, a heterocyclic ring or a cycloalkyl ring and wherein the polycycle comprises any combination of one or more aryl rings, one or more heteroaryl rings, one or more heterocyclic rings, or one or more cycloalkyl rings, with each heterocyclic ring containing from 1 to 5 heteroatoms selected from the group consisting of nitrogen, sulfur, and oxygen;
R_{1b} and R_{2b} are independently H, oxo, OH, OR_{3b}, halogen, CN, NO₂, COOH, NH₂, NHR_{3b}, NR_{3b}R_{4b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; and
R_{3b} and R_{4b} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl containing from 1 to 5 heteroatoms selected from the group consisting of nitrogen, sulfur, and oxygen,
or
(B) formula (II)

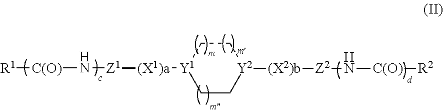

wherein A is —CH₂— or -Q-(CH₂)ₙ—;
each ----- is optionally present, and when present is a single or double bond;
Y¹ and Y² are each independently N or C with the proper valency;
Q is —S—, —O—, or —CH₂—;
X¹ and X² are each independently —NH—, —O—, —CH₂—O—, —NH—CH₂—, or —N(CH₃)—CH₂—;
a and b are each independently 0 or 1;
c and d are each independently 0 or 1;
m, m′, m″, and n are each independently an integer of 0-2;
Z¹ and Z² are each independently a heterocyclic; and
R¹ and R² are each independently optionally substituted alkyl, optionally substituted aralkyl, optionally substituted cycloalkyl, amino, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl;
provided that if c is 0 and d is 0, then R¹ and R² are both amino;
provided that if c is 1 and d is 1, then both R¹ and R² are not amino;
provided that if c is 0 and d is 1, then R¹ is amino and R² is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl; and
provided that if c is 1 and d is 0, then R² is amino and R¹ is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl, or a pharmaceutically acceptable salt of formula (I) or formula (II).

Another aspect of the present disclosure relates a method of inhibiting viral replication. The method comprises contacting one or more cells infected with a virus with an effective amount of a compound of either:
(A) formula (I)

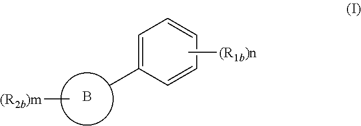

wherein:
m and n are integers from 1 to 4;
B is a substituted or unsubstituted mono or polycycle, wherein the monocycle is an aryl ring, a heteroaryl ring, a heterocyclic ring or a cycloalkyl ring and wherein the polycycle comprises any combination of one or more aryl rings, one or more heteroaryl rings, one or more heterocyclic rings, or one or more cycloalkyl rings, with each heterocyclic ring containing from 1 to 5 heteroatoms selected from the group consisting of nitrogen, sulfur, and oxygen;
R_{1b} and R_{2b} are independently H, oxo, OH, OR_{3b}, halogen, CN, NO₂, COOH, NH₂, NHR_{3b}, NR_{3b}R_{4b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; and
R_{3b} and R_{4b} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl containing from 1 to 5 heteroatoms selected from the group consisting of nitrogen, sulfur, and oxygen,
or
(B) formula (II)

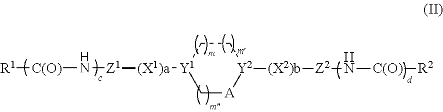

wherein A is —CH₂— or -Q-(CH₂)ₙ—;
each ----- is optionally present, and when present is a single or double bond;
Y¹ and Y² are each independently N or C with the proper valency;
Q is —S—, —O—, or —CH₂—;
X¹ and X² are each independently —NH—, —O—, —CH₂—O—, —NH—CH₂—, or —N(CH₃)—CH₂—;
a and b are each independently 0 or 1;
c and d are each independently 0 or 1;
m, m′, m″, and n are each independently an integer of 0-2;
Z¹ and Z² are each independently a heterocyclic; and
R¹ and R² are each independently optionally substituted alkyl, optionally substituted aralkyl, optionally substituted cycloalkyl, amino, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl;
provided that if c is 0 and d is 0, then R¹ and R² are both amino;
provided that if c is 1 and d is 1, then both R¹ and R² are not amino;
provided that if c is 0 and d is 1, then R¹ is amino and R² is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl; and
provided that if c is 1 and d is 0, then R² is amino and R¹ is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl, or a pharmaceutically acceptable salt of formula (I) or formula (II).

Another aspect of the disclosure relates to a therapeutic composition comprising a compound of formula (I)

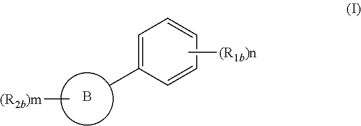

wherein:
m and n are integers from 1 to 4;
B is a substituted or unsubstituted mono or polycycle, wherein the monocycle is an aryl ring, a heteroaryl ring, a heterocyclic ring or a cycloalkyl ring and wherein the polycycle comprises any combination of one or more aryl rings, one or more heteroaryl rings, one or more heterocyclic rings, or one or more cycloalkyl rings, with each heterocyclic ring containing from 1 to 5 heteroatoms selected from the group consisting of nitrogen, sulfur, and oxygen;
R_{1b} and R_{2b} are independently H, oxo, OH, OR_{3b}, halogen, CN, NO₂, COOH, NH₂, NHR_{3b}, NR_{3b}R_{4b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; and
R_{3b} and R_{4b} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl containing from 1 to 5 heteroatoms selected from the group consisting of nitrogen, sulfur, and oxygen, or a pharmaceutically acceptable salt of formula (I).

Developing therapeutic strategies against COVID-19 has gained widespread interest given the likelihood that new viral variants will continue to emerge. The present disclosure describes one potential therapeutic strategy which involves targeting members of the glutaminase family of mitochondrial metabolic enzymes (GLS and GLS2), which catalyze the first step in glutamine metabolism, the hydrolysis of glutamine to glutamate. Three examples are shown, where GLS expression increases during Coronavirus infection of the host cells, and another in which GLS2 is upregulated. The viruses hijack the metabolic machinery responsible for glutamine metabolism to generate the building blocks for biosynthetic processes and satisfy the bioenergetic requirements demanded by the ‘glutamine addiction’ of virus-infected host cells. Genetic silencing of glutaminase enzymes is shown to reduce Coronavirus infection and demonstrate that new members of two classes of small molecule allosteric inhibitors targeting these enzymes, designated as Compound B and Compound D, effectively block GLS-dependent viral replication in mammalian epithelial cells, whereas Compound B is specifically effective against GLS2-dependent virus infection. Overall, these findings highlight the importance of glutamine metabolism for Coronavirus replication in human cells, and that glutaminase inhibitors including newly developed compounds can block Coronavirus infection and thereby may represent a novel class of anti-viral drug candidates.

Herein is shown that three members of the Coronavirus family, SARS-CoV2, HCoV-OC43 and HCoV-229E, reprogram the metabolic machinery of host cells, and in doing so, cause their replication to become glutamine-addicted. In these studies, the mouse kidney epithelial cell line VeroE6 for SARS-CoV2 infections, the human bronchial epithelial cell line HBEC and human colon cancer HCT8 epithelial cells for HCoV-OC43, and HBEC together with the lung epithelial cell line MRC5 for HCoV-229E were used (shown in Table 1). Glutaminase inhibitors, including two new compounds, are demonstrated to represent potential therapeutic agents for blocking Coronavirus infection.

### BRIEF DESCRIPTION OF THE DRAWINGS

FIGS. 1A-1I show GLS expression is increased during Coronavirus infection. FIG. 1A is a Western blot analysis of the cells expressing the viral N-protein OC43, GLS and GLS2 before and after HCoV-OC43 infection (24 h) in HBECs. HBECs that were infected with HCoV-OC43 (MOI 0.01, 1 h at 33° C.) and incubated for 23 hours, and then cells and media were collected. FIG. 1B is Western blot analysis under the same conditions as FIG. 1A, but in HCT8 cells. Total RNA was isolated from the virus-infected cell medium (24 h, 48 h), and qPCR assays showed GLS and GLS2 mRNA levels before and after HCoV-OC43 infections for 24 and 48 hours in HBECs (FIG. 1C) and HCT8 cells (FIG. 1D). FIG. 1E shows co-immunofluorescent staining with HCoV-OC43 and GLS antibodies in HBECs. Cells were infected with the HCoV-OC43 virus for 24 hours, One drop of NucBlue Live Cell stain solution (Invitrogen R37605) was added to the medium per well of 6-well plates 1 hour before the cells were fixed with formaldehyde (3.7%) (a) nuclear staining with blue color, (b) staining with GLS rabbit polyclonal antibody (homemade) and Goat anti-rabbit Alexa Fluor™ 568 Secondary antibody, (c) staining with HCoV-OC43 mouse monoclonal antibody (Millipore, MAB9013) and Goat anti-mouse Alexa Fluor™ 488 Secondary antibody. (d) Merge image of three staining of a,b,c. FIG. 1F shows the same staining from FIG. 1E, applied to HCT8 cells. FIG. 1G is a Western blot analysis showing the time course for GLS and HCoV-OC43 expression levels during virus infection of HBECs. FIG. 1H shows data from qPCR assay performed on the total RNA samples isolated from different time points of virus-infected HBEC cells. FIG. 1I shows data from qPCR assays performed on the total RNA samples isolated from different time points of virus-infected HBEC media.

FIGS. 2A-2D show glutaminase is an essential enzyme for Coronavirus replication. FIG. 2A is a Western blot analysis showing HCoV-OC43 replication levels in HBECs expressing a control shRNA or two independent GLS-targeted shRNAs. FIG. 2B shows data from qPCR assays showing the relative virus RNA levels in the media of control and two GLS-targeted shRNAs. FIG. 2C is a Western blot analysis showing HCoV-OC43 replication levels in HBECs expressing control siRNA or two separated GLS-targeted siRNAs. FIG. 2D shows data from a qPCR analysis showing the total virus RNA levels from the media of non-virus infection, virus-infected cells with control siRNA, and two GLS-targeted siRNA knockdowns. One-way ANOVA with Bonferroni correction was used to determine significance in FIGS. 1B and 1D, **** indicates modified P<0.0001.

FIGS. 3A-B show allosteric inhibitors targeting GLS. FIG. 3A provides the structures of two classes of Glutaminase inhibitors: Compound A and the more potent analog Compound B; and Compound C group and its more potent analog Compound D. FIG. 3B is the Homo-tetramer of human GLS structure showing the docking sites of Compound A group and the binding location of Compound C class.

FIGS. 4A-4F show GLS inhibitors block Coronavirus SARS-CoV-2 and HCoV-OC43 replication. FIG. 4A is a Western blot analysis of cell samples showing the SARS-CoV2 replication levels in cells treated with the different inhibitors compared with DMSO treated virus-infected cells. VeroE6 cells when 70-80% confluence were pretreated with Compound A (2.5 mM), Compound C (2.5 mM), Compound B (1 mM), Compound D (0.1 mM), and DMSO as the control for 3 hours, followed by SARS-CoV2 infection (MOI 0.01) for 1 hour at 33° C. The growth medium was changed with or without inhibitors and the cells were incubated at 37° C. for 23 hours. The media and cells were collected. FIG. 4B is a Western blot analysis showing the OC43 replication levels in cells treated with different inhibitors compared with no drug treatment. HBECs were pretreated with DMSO, Compound A (2.5 mM), Compound C (2.5 mM), Compound B (1 mM), and Compound D (0.1 mM) for 3 hours, followed by HCoV-OC43 infection for 1 hour at 33° C., and then cultured in growth medium with or without inhibitors for 23 hours at 37° C. In FIG. 4C, HCT8 cells were infected with HCoV-OC43 as in FIG. 4B and analyzed for virus replication by Western blotting for OC43. FIG. 4D shows data from qPCR assays showing the relative virus RNA levels in the SARS-CoV 2 infected cell media. FIG. 4E shows data from qPCR assays showing the OC43 virus RNA levels in the media of HCoV-OC43 infected HBECs with or without inhibitors. FIG. 4F shows OC43 virus RNA levels from the medium of HCT8 cells treated with or without inhibitors. One-way ANOVA with Bonferroni correction was used to determine significance in FIGS. 4D-4F, **** indicates modified P<0.0001.

FIGS. 5A-5D show GLS inhibitors do not block Coronavirus entry. FIG. 5A is a diagram showing two sub-classes of human Coronaviruses. HCoV-229E belongs to the alpha group. Beta sub-group contains HCoV-Oc43, HCoV-HUK1, MERS-CoV, SARS-CoV, and SARS-CoV 2. FIG. 5B shows HBECs that were pre-treated with DMSO, Compound A (2.5 mM), Compound C (2.5 mM), Compound B (1 mM), and Compound D (0.1 mM) for 3 hours, then the cells were infected with HCoV-OC43 (MOI 0.01) for 1 hour at 33° C. and the cells were collected. Western blot analysis showing the HCoV-OC43 levels in the HBECs with or without inhibitors. In FIG. 5C, the same analysis in FIG. 5B was performed in HCT8 cells treated with Compound B and Compound D inhibitors. Western blot analysis showing the HCoV-OC43 levels in the cells. In FIG. 5D, HBECs were treated with inhibitors and infected with the virus as above. The total viral RNA levels for each condition were analyzed by qPCR.

FIG. 6 is a schematic of GLS mediated Coronavirus replication. Coronaviruses hijack the metabolic program of host cells and by increasing the expression of GLS. Two possible mechanisms for upregulating GLS involve signaling through c-Myc and c-Jun.

FIGS. 7A-7C show GLS is upregulated through c-Jun and c-Myc regulation during the Coronavirus replication. FIG. 7A is a Western blot analysis showing p-c-Jun(S73), p-c-Jun(S63), c-Jun, p-c-Myc and c-Myc levels are upregulated after HCoV-OC43 infection in HBECs. HBECs were infected with HCoV-OC43 for 24 hours (described previously) with the non-treated cells as a control. The whole cell lysates were collected. FIG. 7B provides data showing that blocking-Jun activation by inhibiting the c-Jun-N-Teminal kinase (INK) reduces GLS expression in virus-infected host cells. FIG. 7C shows data from qPCR assays showing the relative HCoV-OC43 mRNA levels in the media from cells treated with or without JNK inhibitor. One-way ANOVA with Bonferroni correction was used to determine significance in FIG. 7C, **** indicates modified P<0.0001.

FIGS. 8A-8D show dose-dependent inhibition of virus infected HBECs and HCT8 cells by Compound B and Compound D. FIG. 8A is a Western blot analysis showing the effects of Compound B on HCoV-OC43 replication in HBECs. HBECs were pretreated with different doses of Compound B inhibitor (0, 0.5, 1, 2.5, 5, 10 mM) for 3 hours, followed by infection with HCoV-OC43 (MOI 0.01, 2% HS RPMI, 33° C. 5% CO₂ for 1 hour). The cells were changed to the growth media with different concentrations of Compound B and were incubated for 23 hours at 37° C., 5% CO₂. FIG. 8B is Western blot analysis showing the effect of varying doses of Compound D (0, 0.1, 0.5, 1, 5, 10 mM) on a 24-hour virus infection HBECs. FIG. 8C is a Western blot analysis showing the different virus levels as a function of Compound B concentrations. HCT8 cells were pretreated with different concentrations of Compound B for 3 hours and were infected with HCoV-OC43, as described above. FIG. 8D is a Western blot analysis showing the HCoV-OC43 replication levels as a function of different amounts of Compound D. HCT8 cells were pretreated with varying doses of Compound D and then infected with HCoV-OC43.

FIGS. 9A-9B show Compound B and Compound D inhibit HCoV-OC43 replication in HBECs by plaque assays. HBECs (2×10⁵) were seeded in 6-well plates, pretreated with DMSO, Compound B (2.5 μM), and Compound D (0.1 μM) for 3 hours, infected with HCoV-OC43 (MOI 0.1) at 33° C. 5% CO₂ shaking for 1 hour, at which point the agar medium was replaced with the virus solution after a PBS wash. The plates were maintained at room temperature hood for 15 mins and then incubated at 37° C. 5% CO₂ for 4 days. The agar layer was removed, and cells were fixed with 4% PFA and stained with HCoV-OC43 antibody and goat anti-mouse secondary antibody. Triplicate determinations were performed for each condition. FIG. 9A shows imaging of the plaques. FIG. 9B is a bar graph showing the relative plaque numbers for virus-infected cells treated with Compound B or Compound D, DMSO control-treated cells. One-way ANOVA with Bonferroni correction was used to determine significance in FIG. 9B, **** indicates modified P<0.0001.

FIGS. 10A-C show Compound B, but not Compound D, inhibits HCov-229E replication. MRC5 (human fetal lung fibroblast cells) cells were infected with HCoV-229E (MOI 0.01) for 1 h at 34° C. 5% CO₂, and then changed to growth medium and incubated at 37° C. incubator for 23 hours. The cells were collected, and the Western blot analysis in FIG. 10A shows the levels of HCoV-229E (229E), GLS and GLS2. FIG. 10B provides data from qPCR assays showing HCoV-229E mRNA levels in the media of infected MRC5 cells as a function of different doses of Compound D. FIG. 10C provides data from qPCR assays showing the HCoV-229E mRNA levels in the media from infected HBECs treated with different concentrations of Compound B. One-way ANOVA with Bonferroni correction was used to determine no significance in FIG. 10B,*, and significance in FIG. 10C,**** indicates modified P<0.0001.

FIGS. 11A-B show that Compound B inhibits feline Coronavirus FIPV 1 Black replication. FIG. 11A provides data from a plaque assay. FCWF-4 CU cells (Animal Health Diagnostic Center, Cornell University) were grown in the 75 cm flasks until 80% confluence, and pre-treated the cells with either DMSO, or Compound B (5 uM) for 24 hours. The cells were infected with FIPV 1 Black (MOI 0.01) for 1 hour, and then the cells of each condition were changed to cell growth medium containing either DMSO, Compound B (5 uM) or GS-44154 (it was used as a positive control, it is an anti-Covid-19 drug) for 23 hours. The media containing the viruses were collected. Serial dilutions of each viral medium were made and applied to FCWF-4 CU cells in 12-well plates. 4×10⁵ FCWF-4 CU cells per well were grown on 12-well plates, each dilution was applied and duplicated, and the plates were incubated at 37° C., 5% CO₂ for 3 days. The plates were stained with crystal violet (0.1%) after being fixed with 4% PFA, washed with water until the plaques were visualized. The plaques of each condition were counted and the numbers of 10⁴ dilution wells were shown and graphed. FIG. 11B is a Western blot analysis showing that 1 μM Compound B reduces the production of feline viral protein. FCWF-4-CU cells were grown in 75 cm flasks until 80% confluence, pre-treated with DMSO or Compound B (5 μM) for 24 hours, then the cells were infected with FIPV 1 Black (MOI 0.01) for 1 hour, post-treated with DMSO or Compound B in growth medium for 23 hours. The cells were collected from each condition, and a Western blot assay was performed with an anti-feline coronavirus antibody FIPV3-70 (SANTA CRUZ, cat #sc-65653) and an anti-Vinculin antibody as the loading control (SANTA CRUZ, cat #sc-73614).

### DETAILED DESCRIPTION

Unless otherwise indicated, the definitions and embodiments described in this and other sections are intended to be applicable to all embodiments and aspects of the present application herein described for which they are suitable as would be understood by a person skilled in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “and/or” as used herein means that the listed items are present, or used, individually or in combination. In effect, this term means that “at least one of” or “one or more” of the listed items is used or present.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of” The methods, kits, systems, and/or compositions of the present disclosure can comprise, consist essentially of, or consist of, the components disclosed.

In embodiments comprising an “additional” or “second” component, the second component as used herein is different from the other components or first component. A “third” component is different from the other, first, and second components, and further enumerated or “additional” components are similarly different.

Certain terms employed in the specification, examples, and claims are collected herein. Unless defined otherwise, all technical and scientific terms used in this disclosure have the same meanings as commonly understood by one of ordinary skill in the art to which this disclosure belongs.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

A first aspect of the disclosure relates to a method of treating a virus infection in a subject. The method comprises administering to the subject a compound of either: (A) formula (I)

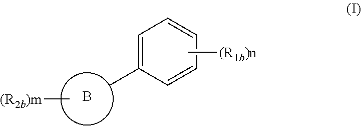

wherein.
m and n are integers from 1 to 4;
B is a substituted or unsubstituted mono or polycycle, wherein the monocycle is an aryl ring, a heteroaryl ring, a heterocyclic ring or a cycloalkyl ring and wherein the polycycle comprises any combination of one or more aryl rings, one or more heteroaryl rings, one or more heterocyclic rings, or one or more cycloalkyl rings, with each heterocyclic ring containing from 1 to 5 heteroatoms selected from the group consisting of nitrogen, sulfur, and oxygen;
R_{1b} and R_{2b} are independently H, oxo, OH, OR_{3b}, halogen, CN, NO₂, COOH, NH₂, NHR_{3b}, NR_{3b}R_{4b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; and
R_{3b} and R_{4b} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl containing from 1 to 5 heteroatoms selected from the group consisting of nitrogen, sulfur, and oxygen,
or
(B) formula (II)

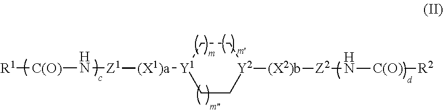

wherein A is —CH₂— or -Q-(CH₂)ₙ—;
each ----- is optionally present, and when present is a single or double bond;
Y¹ and Y² are each independently N or C with the proper valency;
Q is —S—, —O—, or —CH₂—;
X¹ and X² are each independently —NH—, —O—, —CH₂—O—, —NH—CH₂—, or —N(CH₃)—CH₂—;
a and b are each independently 0 or 1;
c and d are each independently 0 or 1;
m, m′, m″, and n are each independently an integer of 0-2;
Z¹ and Z² are each independently a heterocyclic; and
R¹ and R² are each independently optionally substituted alkyl, optionally substituted aralkyl, optionally substituted cycloalkyl, amino, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl;
provided that if c is 0 and d is 0, then R¹ and R² are both amino;
provided that if c is 1 and d is 1, then both R¹ and R² are not amino;
provided that if c is 0 and d is 1, then R¹ is amino and R² is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl; and
provided that if c is 1 and d is 0, then R² is amino and R¹ is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl,
or a pharmaceutically acceptable salt of formula (I) or formula (II).

Another aspect of the present disclosure relates a method of inhibiting viral replication. The method comprises contacting one or more cells infected with a virus with an effective amount of a compound of either:
(A) formula (I)

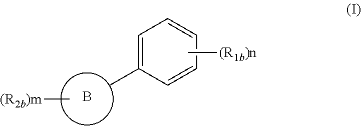

wherein:
m and n are integers from 1 to 4; B is a substituted or unsubstituted mono or polycycle, wherein the monocycle is an aryl ring, a heteroaryl ring, a heterocyclic ring or a cycloalkyl ring and wherein the polycycle comprises any combination of one or more aryl rings, one or more heteroaryl rings, one or more heterocyclic rings, or one or more cycloalkyl rings, with each heterocyclic ring containing from 1 to 5 heteroatoms selected from the group consisting of nitrogen, sulfur, and oxygen;
R_{1b} and R_{2b} are independently H, oxo, OH, OR_{3b}, halogen, CN, NO₂, COOH, NH₂, NHR_{3b}, NR_{3b}R_{4b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; and
R_{3b} and R_{4b} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl containing from 1 to 5 heteroatoms selected from the group consisting of nitrogen, sulfur, and oxygen,
or
(B) formula (II)

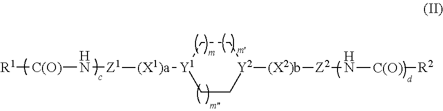

wherein A is —CH₂— or -Q-(CH₂)ₙ—;
each ----- is optionally present, and when present is a single or double bond;
Y¹ and Y² are each independently N or C with the proper valency;
Q is —S—, —O—, or —CH₂—;
X¹ and X² are each independently —NH—, —O—, —CH₂—O—, —NH—CH₂—, or —N(CH₃)—CH₂—;
a and b are each independently 0 or 1;
c and d are each independently 0 or 1;
m, m′, m″, and n are each independently an integer of 0-2;
Z¹ and Z² are each independently a heterocyclic; and
R¹ and R² are each independently optionally substituted alkyl, optionally substituted aralkyl, optionally substituted cycloalkyl, amino, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl;
provided that if c is 0 and d is 0, then R¹ and R² are both amino;
provided that if c is 1 and d is 1, then both R¹ and R² are not amino;
provided that if c is 0 and d is 1, then R¹ is amino and R² is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl; and
provided that if c is 1 and d is 0, then R² is amino and R¹ is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl,
or a pharmaceutically acceptable salt of formula (I) or formula (II).

The term “halo” or “halogen” means fluoro, chloro, bromo, or iodo.

The term “optionally substituted” indicates that a group may have a substituent at each substitutable atom of the group (including more than one substituent on a single atom), and the identity of each substituent is independent of the others.

The term “substituted” or “substitution” of an atom means that one or more hydrogen on the designated atom is replaced with a selection from the indicated group, provided that the designated atom's normal valency is not exceeded. “Unsubstituted” atoms bear all of the hydrogen atoms dictated by their valency. When a substituent is oxo (i.e., ═O), then 2 hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent. Unless otherwise defined, the term “optionally-substituted” or “optional substituent” as used herein refers to a group which may or may not be further substituted with 1, 2, 3, 4 or more groups, or 1, 2 or 3, or 1 or 2 groups. Exemplary substituents include, without limitation, oxo, thio (i.e. ═S), nitro, cyano, halo, OH, NH₂, C₁-C₆ alkyl, C₁-C₆ alkoxy, C₂-C₆ alkenyl, C₂-C₆ alkynyl, C₃-C₆ cycloalkyl, C₄-C₇ cycloalkylalkyl, monocyclic aryl, monocyclic hetereoaryl, polycyclic aryl, polycyclic heteroaryl, aryloxy, C₁₋₆ alkoxyaryl, C₁₋₆ alkylhalo (such as CF₃ and CHF₂), C₁₋₆ alkoxyhalo (such as OCF₃ and OCHF₂), carboxyl, esters, cyano, amino, substituted amino, disubstituted amino, acyl, ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC₁₋₆alkyl, heterocyclyl wherein each alkyl, alkenyl, alkynyl, cycloalkyl, aryl and heterocyclyl and groups containing them may be further optionally substituted. Optional substituents in the case N-heterocycles may also include but are not limited to C₁₋₆ alkyl and/or methyl, for example N-methyl.

The term “monocyclic” indicates a molecular structure having one ring.

The term “polycyclic” indicates a molecular structure having two or more rings, including, but not limited to, fused, bridged, or spiro rings.

The term “alkyl” means an aliphatic hydrocarbon group which may be straight or branched having about 1 to about 24 carbon atoms in the chain. In one example, the alkyl may be straight or branched having about 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl.

A “lower alkyl” group may include a saturated branched or unbranched hydrocarbon having from 1 to 6 carbon atoms. Alkyl groups may have 1 to 4 carbon atoms. Alkyl groups may be “substituted alkyls” wherein one or more hydrogen atoms are substituted with a substituent. Examples of substituents include, but are not limited to, halogen, cycloalkyl, alkoxy, amino, hydroxyl, aryl, heteroaryl, heterocycloalkyl, alkenyl, carboxyl, etc. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl (C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, ally!, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆) alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

The term “alkenyl” means an aliphatic hydrocarbon group containing a carbon carbon double bond and which may be straight or branched having about 2 to about 6 carbon atoms in the chain. Alkenyl groups may have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. Alkenyl groups may be unsubstituted or substituted. “Lower alkenyl” groups may contain one to six carbon atoms.

The term “alkynyl” means an aliphatic hydrocarbon group containing a carbon carbon triple bond and which may be straight or branched having about 2 to about 12 carbon atoms in the chain. In one example, the alkynyl may have about 2 to about 6 carbon atoms in the chain. In one example, alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl. Alkynyl groups may be unsubstituted or substituted. “Lower alkynyl” groups may be those that contain one to six carbon atoms.

The term “alkoxy” refers to an alkyl-O—, alkenyl-O—, or alkynyl-O— group wherein the alkyl, alkenyl, or alkynyl group is described above. “Alkoxy” may refer to a straight, branched or cyclic hydrocarbon configuration and combinations thereof, including from 1 to 20 carbon atoms, for example, from 1 to 8 carbon atoms (referred to as a “lower alkoxy”), or from 1 to 4 carbon atoms, which include an oxygen atom at the point of attachment. An example of an “alkoxy group” is represented by the formula —OR, where R can be an alkyl group, optionally substituted with an alkenyl, alkynyl, aryl, heteroaryl, aralkyl, heteroaralkyl, cycloalkyl, halogenated alkyl, alkoxy or heterocycloalkyl group. Exemplary alkoxy groups include methoxy, ethoxy, n-propoxy, i-propoxy, n-butoxy, pentoxy, hexoxy, i-butoxy, sec-butoxy, tert-butoxy cyclopropoxy, cyclohexyloxy, and the like.

The term “alkoxycarbonyl” refers to an alkoxy substituted carbonyl radical, C (O)OR, wherein R represents an optionally substituted alkyl, aryl, aralkyl, cycloalkyl, cycloalkylalkyl or similar moiety.

“Carbonyl” refers to a radical of the formula —C(O)·Carbonyl-containing groups include any substituent containing a carbon-oxygen double bond (C═O), including acyl groups, amides, carboxy groups, esters, ureas, carbamates, carbonates and ketones and aldehydes, such as substituents based on —COR or —RCHO where R is an aliphatic, heteroaliphatic, alkyl, heteroalkyl, hydroxyl, or a secondary, tertiary, or quaternary amine.

The term “carboxylate” or “carboxyl” refers to the group —COO⁻ or —COH. The carboxyl group can form a carboxylic acid. “Substituted carboxyl” refers to —COOR where R is alkyl, alkenyl, alkynyl, aryl, aralkyl, cycloalkyl, halogenated alkyl, or heterocycloalkyl group. For example, a substituted carboxyl group may be a carboxylic acid ester or a salt thereof (e.g., a carboxylate).

The term “cycloalkyl” refers to a non-aromatic saturated or unsaturated mono- or polycyclic ring system which may contain 3 to 6 carbon atoms; and which may include at least one double bond. Exemplary cycloalkyl groups include, without limitation, cyclopropyl, cyclobutyl, cyclopentyl, cyclohexyl, cyclopropenyl, cyclobutenyl, cyclopentenyl, cyclohexenyl, anti-bicyclopropane, or syn-bicyclopropane.

The term “cycloalkylalkyl” refers to a radical of the formula —R^{a}R^{b} where R^{a} is an alkyl radical as defined above and R^{b} is a cycloalkyl radical as defined herein. The alkyl radical and the cycloalkyl radical may be optionally substituted as defined above.

The term “aryl” refers to aromatic monocyclic or polycyclic ring system containing from 6 to 19 carbon atoms, where the ring system may be optionally substituted. Aryl groups of the present disclosure include, but are not limited to, groups such as phenyl, naphthyl, azulenyl, phenanthrenyl, anthracenyl, fluorenyl, pyrenyl, triphenylenyl, chrysenyl, and naphthacenyl, which can be optionally substituted.

The term “arylalkyl” refers to a radical of the formula —R^{a}R^{b} where R^{a} is an alkyl radical as defined above and R^{b} is an aryl radical as defined above. The alkyl radical and the cycloalkyl radical may be optionally substituted as defined above.

The term “arylarylalkyl” refers to a radical of the formula —R^{a}R^{b}R^{c} where R^{a} is an alkyl as defined above, R^{b} is an aryl radical as defined above, and R_{c} is an aryl radical as defined above. The alkyl radical and both aryl radicals may be optionally substituted as defined above.

The term “aralkyl” refers to an alkyl group wherein an aryl group is substituted for a hydrogen of the alkyl group. An example of an aralkyl group is a benzyl group.

The terms “aryloxy” or “heteroaryloxy” refer to a group of the formula OAr, wherein Ar is an aryl group or a heteroaryl group, respectively.

The term “hydroxyl” is represented by the formula —OH. The term “hydroxyalkyl” refers to an alkyl group that has at least one hydrogen atom substituted with a hydroxyl group. The term “alkoxyalkyl group” is defined as an alkyl group that has at least one hydrogen atom substituted with an alkoxy group described above.

The term “heterocyclyl” refers to a stable 3- to 18-membered ring radical which consists of carbon atoms and from one to five heteroatoms selected from the group consisting of nitrogen, oxygen and sulfur. For purposes of this disclosure, the heterocyclyl radical may be a monocyclic, or a polycyclic ring system, which may include fused, bridged, or spiro ring systems; and the nitrogen, carbon, or sulfur atoms in the heterocyclyl radical may be optionally oxidized; the nitrogen atom may be optionally quaternized; and the ring radical may be partially or fully saturated. Examples of such heterocyclyl radicals include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone.

The term “heterocyclic” refers to a closed-ring compound, or radical thereof as a substituent bonded to another group, particularly other organic groups, where at least one atom in the ring structure is other than carbon, and typically is oxygen, sulfur and/or nitrogen.

The term “heterocycloalkyl group” is a cycloalkyl group as defined above where at least one of the carbon atoms of the ring is replaced by a heteroatom such as, but not limited to, nitrogen, oxygen, sulfur, or phosphorous.

The term “ester” refers to a carboxyl group-containing moiety having the hydrogen replaced with, for example, a C₁₋₆ alkyl group (“carboxylC₁₋₆alkyl” or “alkylester”), an aryl or aralkyl group (“arylester” or “aralkylester”) and so on. CO₂C₁₋₃ alkyl groups may be present, such as for example, methylester (CO₂Me), ethylester (CO₂Et) and propylester (CO₂Pr) and includes reverse esters thereof (e.g., OCOMe, OCOEt and OCOPr).

The terms “halogenated alkyl” or “haloalkyl group” refer to an alkyl group with one or more hydrogen atoms present on these groups substituted with a halogen (F, Cl, Br, I). The term “heteroaralkyl” refers to an alkyl group wherein a heteroaryl group is substituted for a hydrogen in the said alkyl group.

The term “heteroaryl” refers to an aromatic ring radical which consists of carbon atoms and from one to five heteroatoms selected from the group consisting of nitrogen, oxygen, and sulfur. For purposes of this disclosure the heteroaryl may be a monocyclic or polycyclic ring system; and the nitrogen, carbon, and sulfur atoms in the heteroaryl ring may be optionally oxidized; the nitrogen may optionally be quaternized. Examples of heteroaryl groups include, without limitation, pyrrolyl, pyrazolyl, imidazolyl, triazolyl, furyl, thiophenyl, oxazolyl, isoxazolyl, thiazolyl, isothiazolyl, oxadiazolyl, thiadiazolyl, pyridyl, pyrazinyl, pyrimidinyl, pyridazinyl, triazinyl, thienopyrrolyl, furopyrrolyl, indolyl, azaindolyl, isoindolyl, indolinyl, indolizinyl, indazolyl, benzimidazolyl, imidazopyridinyl, benzotriazolyl, benzoxazolyl, benzoxadiazolyl, benzothiazolyl, pyrazolopyridinyl, triazolopyridinyl, thienopyridinyl, benzothiadiazolyl, benzofuyl, benzothiophenyl, quinolinyl, isoquinolinyl, tetrahydroquinolyl, tetrahydroisoquinolyl, cinnolinyl, quinazolinyl, quinolizilinyl, phthalazinyl, benzotriazinyl, chromenyl, naphthyridinyl, acrydinyl, phenanzinyl, phenothiazinyl, phenoxazinyl, pteridinyl, and purinyl.

Further heterocycles and heteraryls are described in Katritzky et al., eds., “Comprehensive Heterocyclic Chemistry: The Structure, Reactions, Synthesis and Use of Heterocyclic Compounds,” Vol. 1-8, Pergamon Press, N.Y. (1984), which is hereby incorporated by reference in its entirety.

The term “n-heterocyclic” refers to mono or bicyclic rings or ring systems that include at least one nitrogen heteroatom. The rings or ring systems may include 1 to 9 carbon atoms in addition to the heteroatom(s) and may be saturated, unsaturated or aromatic (including pseudoaromatic). The term “pseudoaromatic” refers to a ring system which is not strictly aromatic, but which is stabilized by means of delocalization of electrons and behaves in a similar manner to aromatic rings. Aromatic includes pseudoaromatic ring systems, such as pyrrolyl rings.

Examples of 5-membered monocyclic N-heterocycles include pyrrolyl, H-pyrrolyl, pyrrolinyl, pyrrolidinyl, oxazolyl, oxadiazolyl, (including 1,2,3 and 1,2,4 oxadiazolyls) isoxazolyl, furazanyl, thiazolyl, isothiazolyl, pyrazolyl, pyrazolinyl, pyrazolidinyl, imidazolyl, imidazolinyl, triazolyl (including 1,2,3 and 1,3,4 triazolyls), tetrazolyl, thiadiazolyl (including 1,2,3 and 1,3,4 thiadiazolyls), and dithiazolyl. Examples of 6-membered monocyclic N-heterocycles include pyridyl, pyrimidinyl, pyridazinyl, pyrazinyl, piperidinyl, morpholinyl, thiomorpholinyl, piperazinyl, and triazinyl. The heterocycles may be optionally substituted with a broad range of substituents, and with C₁₋₆ alkyl, C₁₋₆ alkoxy, C₂₋₆ alkenyl, C₂₋₆ alkynyl, halo, hydroxy, mercapto, trifluoromethyl, amino, cyano or mono or di(C₁₋₆alkyl)amino. The N-heterocyclic group may be fused to a carbocyclic ring such as phenyl, naphthyl, indenyl, azulenyl, fluorenyl, and anthracenyl.

The term “acyl” refers to a group having the structure —C(O)R, where R may be, for example, optionally substituted alkyl, optionally substituted aryl, or optionally substituted heteroaryl. The term “lower acyl groups may include those that contain one to six carbon atoms.

The term “acyloxy” refers to a group having the structure —OC(O)R—, where R may be, for example, optionally substituted alkyl, optionally substituted aryl, or optionally substituted heteroaryl. The term “lower acyloxy” groups may contain one to six carbon atoms.

The terms “alkanediyl,” “cycloalkanediyl,” “aryldiyl,” and “alkanearyldiyl” refer to a divalent radical derived from aliphatic, cycloaliphatic, aryl, and alkanearyl hydrocarbons.

The term “amine” or “amino” refers to a group of the formula —NRR′, where R and R′ can be, independently, hydrogen or an alkyl, alkenyl, alkynyl, aryl, aralkyl, cycloalkyl, halogenated alkyl, or heterocycloalkyl group. For example, an “alkylamino” or “alkylated amino” refers to —NRR′, wherein at least one of R or R′ is an alkyl.

The term “aminoalkyl” refers to alkyl groups as defined above where at least one hydrogen atom is replaced with an amino group (e.g, —CH₂—NH₂). “Aminocarbonyl” alone or in combination, means an amino substituted carbonyl (carbamoyl) radical, wherein the amino radical may optionally be mono- or di-substituted, such as with alkyl, aryl, aralkyl, cycloalkyl, cycloalkylalkyl, alkanoyl, alkoxycarbonyl, aralkoxycarbonyl and the like. An aminocarbonyl group may be —N(R)—C(O)—R (wherein R is a substituted group or H). A suitable aminocarbonyl group is acetamido.

The term “amide” or “amido” is represented by the formula C(O)NRR′, where Rand R′ independently can be a hydrogen, alkyl, alkenyl, alkynyl, aryl, aralkyl, heteroaryl, heteroaralkyl, cycloalkyl, halogenated alkyl, or heterocycloalkyl group.

An “analog” is a molecule that differs in chemical structure from a parent compound, for example a homolog (differing by an increment in the chemical structure or mass, such as a difference in the length of an alkyl chain or the inclusion of one of more isotopes), a molecular fragment, a structure that differs by one or more functional groups, or a change in ionization. An analog is not necessarily synthesized from the parent compound. A derivative is a molecule derived from the base structure.

The term “compounds of the present disclosure”, and equivalent expressions are meant to embrace compounds of general Formulae (I) and/or (II) (as well as compounds comprising their moieties) as herein before described, which expression includes the prodrugs, the pharmaceutically acceptable salts, and the solvates, e.g., hydrates, where the context so permits. Similarly, reference to intermediates, whether or not they themselves are claimed, is meant to embrace their salts and solvates, where the context so permits. For the sake of clarity, particular instances, when the context so permits, are sometimes indicated in the text, but these instances are purely illustrative and it is not intended to exclude other instances when the context so permits. In one embodiment, the compound used in the method is a compound of formula (I). In another embodiment, the compound used in the method is a compound of formula (II).

In one embodiment, formula (I) comprises formula (Ia):

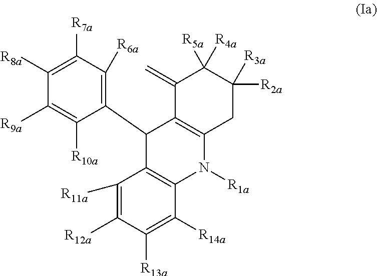

wherein.
R₁ₐ is independently H, OH, OR₁₅ₐ, C₁-C₆ alkyl, C₂-C₆ alkenyl, C₂-C₆ alkynyl, R₁₅ₐC(O)—, R₁₅ₐOC(O)—, R₁₅ₐS(O)—, or R₁₅ₐS(O)₂—;
R₂ₐ, R₃ₐ, R₄ₐ, and R₅ₐ are each independently H, OH, NH₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the aryl, heteroaryl, and aryl C₁-C₆ alkyl, are optionally substituted from 1 to 3 times with substituents independently selected from the group consisting of halogen, OH, NH₂, C₁-C₆ alkyl, C₂-C₆ alkenyl, C₁-C₆ alkoxy, SH, and C₁-C₆ thioalkyl;
R₆ₐ, R₇ₐ, R₈ₐ, R₉ₐ, and R₁₀ₐ are each independently H, halogen, NO₂, OH, OR₁₅ₐ, —SR₁₅ₐ, NH₂, NHR₁₅ₐ, NR₁₅ₐR₁₆ₐ, R₁₅ₐC(O)—, R₁₅ₐOC(O)—, R₁₅ₐC(O)O—, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; or
R₆ₐ and R₇ₐ, R₇ₐ and R₈ₐ, R₈ₐ and R₉ₐ, or R₉ₐ and R₁₀ₐ can combine to form a heterocyclic ring; and
R₁₁ₐ, R₁₂ₐ, R₁₃ₐ, R₁₄ₐ, R₁₅ₐ, and R₁₆ₐ are each independently H, halogen, OH, NO₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, each one of R₁₁ₐ-R₁₆ₐ optionally independently substituted with NH₂, OH, halogen, COOH, NO₂, and CN; or R₁₁ₐ and R₁₂ₐ, R₁₂ₐ and R₁₃ₐ, or R₁₃ₐ and R₁₄ₐ can combine to form an optionally substituted aromatic ring.

Splice isoforms, namely kidney-type glutaminase (KGA) and isoform C (GAC) of the GIs gene (gene locus 2q32-q34) represent the translated forms of the human kidney-type glutaminase or GLS, an enzyme found in abundance in proliferating cells, immune cells, kidney, brain, muscle, and other tissues, and is generally referred to here throughout as GLS. GIs gene products are involved in the hydrolysis of glutamine to glutamate and ammonium.

In some embodiments, the compound of formula (I) may be, but is not limited to, one of the following:

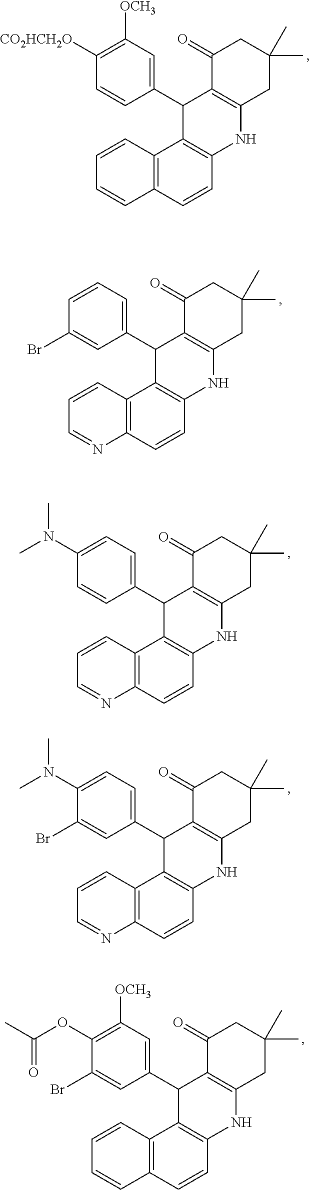

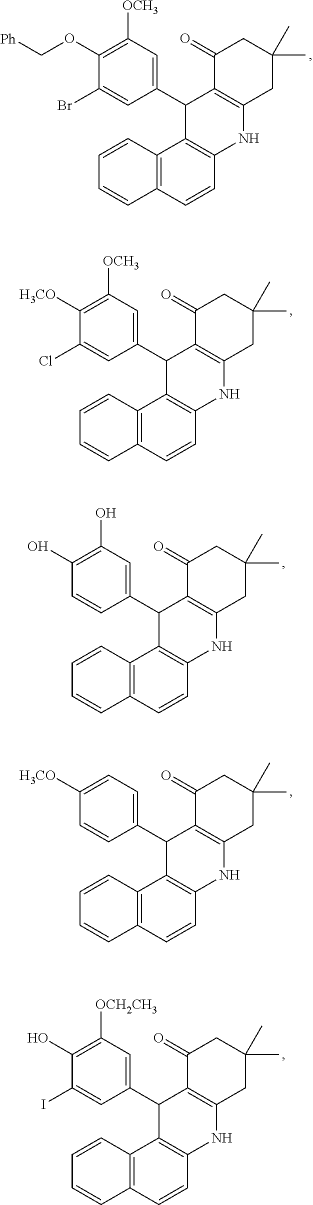 [SMILES: COc1cc(C2C3=C(CC(C)(C)CC3=O)Nc3ccc4ccccc4c32)cc(Br)c1OCc1ccccc1 ; COc1cc(C2C3=C(CC(C)(C)CC3=O)Nc3ccc4ccccc4c32)cc(Cl)c1OC ; CC1(C)CC(=O)C2=C(C1)Nc1ccc3ccccc3c1C2c1ccc(O)c(O)c1 ; COc1ccc(C2C3=C(CC(C)(C)CC3=O)Nc3ccc4ccccc4c32)cc1 ; CCOc1cc(C2C3=C(CC(C)(C)CC3=O)Nc3ccc4ccccc4c32)cc(I)c1O]

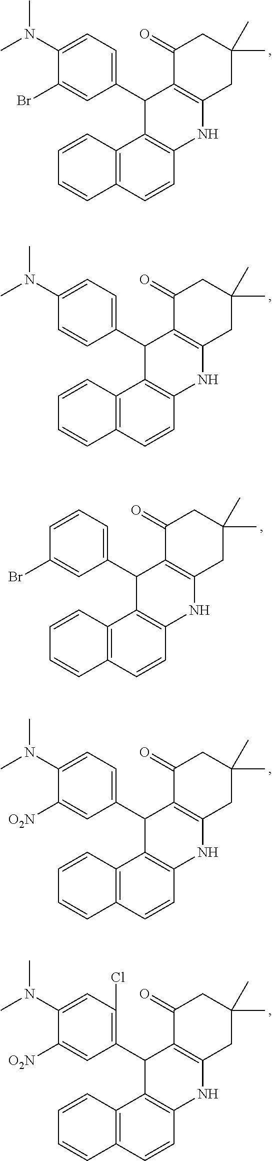

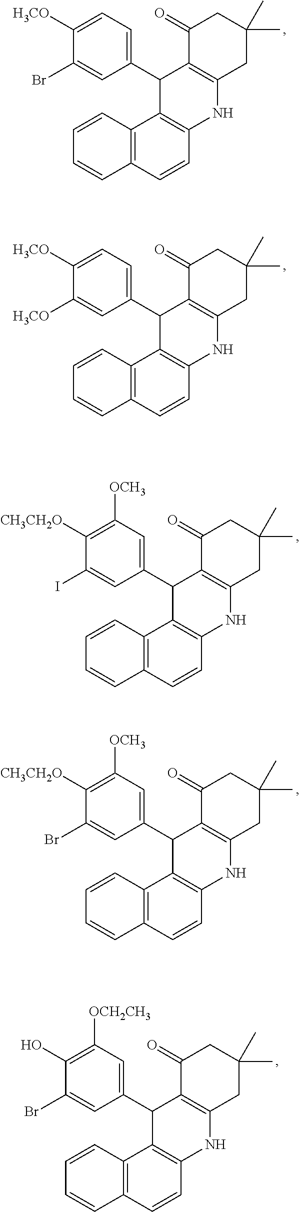

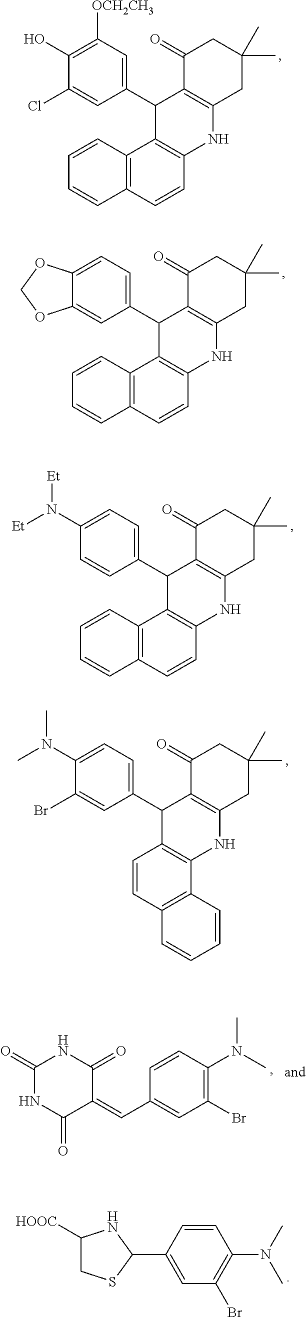

In one embodiment, the compound of formula (I) is

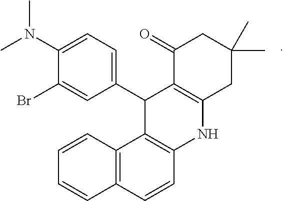

In another embodiment, the compound of formula (I) is

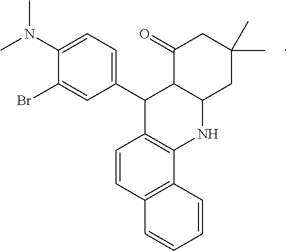

In one embodiment, the compound is a compound of formula (II). In one embodiment, A in formula (II) is —S—(CH₂)ₙ—. In one embodiment, formula (II) comprises formula (IIa):

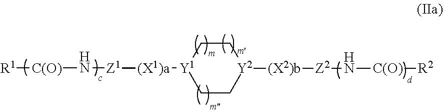

wherein
X¹ and X² are each independently —NH—, —O—, —CH₂—O—, —NH—CH₂—, or —N(CH₃)—CH₂—, provided that when at least one of X¹ and X² is —CH₂—O—, —NH—CH₂—, or —N(CH₃)—CH₂—, then the —CH₂— is directly connected to

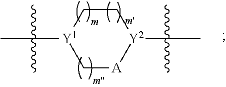

provided that if Y¹ and Y² are each C, then a is 1 and b is 1;
provided that if Y¹ and Y² are each N, then a is 0 and b is 0;
provided that if Y¹ is N and Y² is C, then a is 0 and b is 1; and
provided that if Y¹ is C and Y² is N, then a is 1 and b is 0.

In one embodiment,

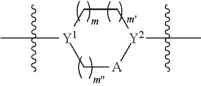

of formula (IIa) is a 3, 4, 5, 6 or 7 membered N-heterocycle. In another embodiment,

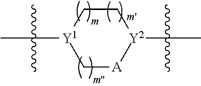

of formula (IIa) is piperidinyl. In another embodiment,

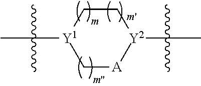

of formula (IIa) is selected from the group consisting of:

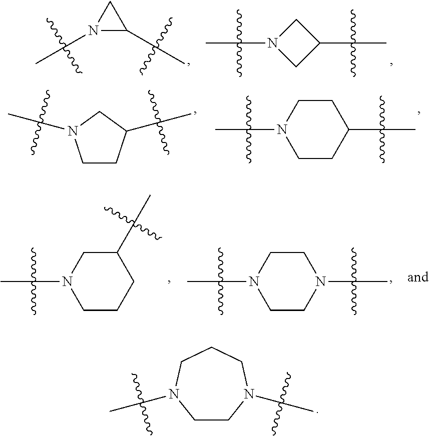

In another embodiment,

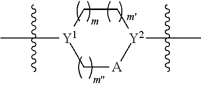

of formula (IIa) is a 3, 4, 5, 6 or 7 membered cycloalkylene where Y¹ and Y² are carbon.

In one embodiment,

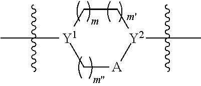

of formula (IIa) is selected from the group consisting of;

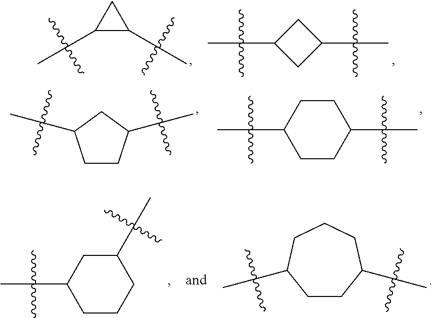

In one embodiment, the compound of formula II is selected from the group consisting of:

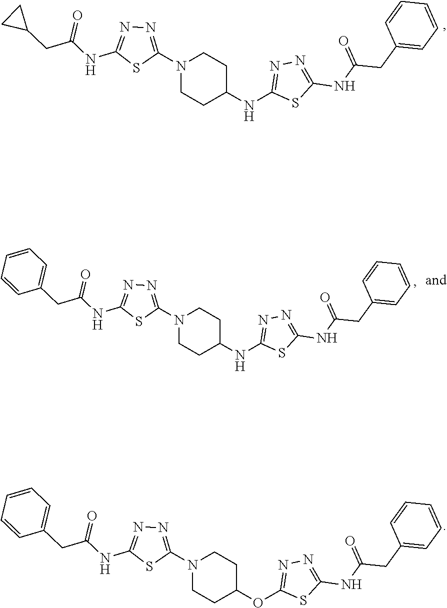 [SMILES: O=C(Cc1ccccc1)Nc1nnc(NC2CCN(c3nnc(NC(=O)CC4CC4)s3)CC2)s1 ; O=C(Cc1ccccc1)Nc1nnc(NC2CCN(c3nnc(NC(=O)Cc4ccccc4)s3)CC2)s1 ; O=C(Cc1ccccc1)Nc1nnc(OC2CCN(c3nnc(NC(=O)Cc4ccccc4)s3)CC2)s1]

In one embodiment, the compound of formula (II) is:

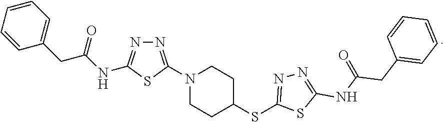 [SMILES: O=C(Cc1ccccc1)Nc1nnc(SC2CCN(c3nnc(NC(=O)Cc4ccccc4)s3)CC2)s1]

In another embodiment, the compound of formula (II) is:

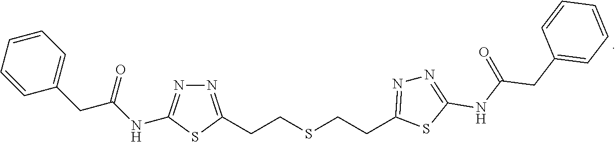 [SMILES: O=C(Cc1ccccc1)Nc1nnc(CCSCCc2nnc(NC(=O)Cc3ccccc3)s2)s1]

In one embodiment, Z¹ and Z² of formula (I) or formula (II) are independently selected from the group consisting of

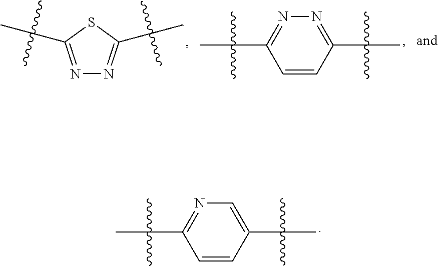 [SMILES: *c1nnc(*)s1 ; *c1ccc(*)nn1 ; *c1ccc(*)nc1]

The methods described herein may include compositions with a “pharmaceutically acceptable inert carrier,” and this expression is intended to include one or more inert excipients, which include, for example and without limitation, starches, polyols, granulating agents, microcrystalline cellulose, diluents, lubricants, binders, disintegrating agents, and the like. If desired, tablet dosages of the disclosed compositions may be coated by standard aqueous or nonaqueous techniques. “Pharmaceutically acceptable carrier” also encompasses controlled release means.

For therapeutic use (e.g., in the method of the present aspect), salts of the compounds are those wherein the counter-ion is pharmaceutically acceptable. However, salts of acids and bases which are non-pharmaceutically acceptable may also find use, for example, in the preparation or purification of a pharmaceutically acceptable compound. The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g., hydrochloric or hydrobromic acid, sulfuric, nitric, phosphoric and the like acids; or organic acids such as, for example, acetic, propanoic, hydroxyacetic, lactic, pyruvic, oxalic (i.e., ethanedioic), malonic, succinic (i.e., butanedioic acid), maleic, fumaric, malic (i.e., hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form. The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g., the lithium, sodium, potassium, magnesium, calcium salts and the like, salts with organic bases, e.g., the benzathine, N-methyl-D-glucamine, hydrabamine salts, and salts with amino acids such as, for example, arginine, lysine and the like.

The term “addition salt” as used herein also comprises the solvates which the compounds described herein are able to form. Such solvates are for example hydrates, alcoholates and the like.

Prodrugs of the disclosed compounds used in the method of the present aspect are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. For a general discussion of prodrugs involving esters see Svensson and Tunek, Drug Metabolism Reviews 165 (1988) and Bundgaard, Design of Prodrugs, Elsevier (1985), which are hereby incorporated by reference in their entirety.

The term “prodrug” also is intended to include any covalently bonded carriers that release an active parent drug of the present disclosure in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group. In particular examples, a prodrug is a lower alkyl phosphonate ester, such as an isopropyl phosphonate ester.

Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999, which is hereby incorporated by reference in its entirety. In general, protecting groups are removed under conditions that will not affect the remaining portion of the molecule. These methods are well known in the art and include acid hydrolysis, hydrogenolysis and the like. For example, the removal of an ester, such as cleavage of a phosphonate ester under Lewis acidic conditions, mediated by TMS-Br to yield the free phosphonate. Removal of a benzyl group may be affected by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known, and suitable protecting groups can be selected by those of skill in the art in consultation with Greene and Wuts, Protective Groups in Organic Synthesis; 3rd Ed.; John Wiley & Sons, New York, 1999, which is hereby incorporated by reference in its entirety, or other chemical literature sources. When an amine is deprotected, the resulting salt can readily be neutralized to yield the free amine. Similarly, when an acid moiety, such as a phosphonic acid moiety is unveiled, the compound may be isolated as the acid compound or as a salt thereof.

“Isomer” refers to one of two or more molecules having the same number and kind of atoms but differing in the arrangement or configuration of the atoms. Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−) isomers respectively). A chiral compound can exist as either individual enantiomer or as a mixture thereof. A mixture containing equal proportions of the enantiomers is called a “racemic mixture.” E/Z isomers are isomers that differ in the stereochemistry of a double bond. An E isomer has a trans-configuration at the double bond, in which the two groups of highest priority are on opposite sides of the double bond. A Z isomer has a cis-configuration at the double bond, in which the two groups of highest priority are on the same side of the double bond.

One aspect of the disclosure as described herein relates to a method of treating a virus infection by administering to a subject a compound of either formula (I), formula (II), or a pharmaceutically acceptable salt thereof.

The terms “treat”, “treating”, “treatment”, and grammatical variations thereof mean subjecting an individual subject to a protocol, regimen, process, or remedy, in which it is desired to obtain a physiologic response or outcome in that subject, e.g., a patient. In particular, the methods and compositions of the present disclosure may be used to slow the development of disease symptoms or delay the onset of the disease or condition or halt the progression of disease development. However, because every treated subject may not respond to a particular treatment protocol, regimen, process, or remedy, treating does not require that the desired physiologic response or outcome be achieved in each and every subject or subject, e.g., patient, population. Accordingly, a given subject or subject, e.g., patient, population may fail to respond or respond inadequately to treatment.

The term “subject” refers to any mammalian subject for whom diagnosis, treatment, or therapy is desired, particularly humans.

In some embodiments, the subject is a mammalian subject. The terms “mammal” or “mammalian subject” for purposes of the methods described herein refers to any animal classified as a mammal, including humans, domestic and farm animals, and zoo, sports, or pet animals, such as felines, dogs, horses, cows, sheep, goats, pigs, camels, etc.

In some embodiments, the mammalian subject is a human subject. The human subject may be an infant, a child, an adolescent, an adult, or a geriatric subject.

In some embodiments, the methods of the present disclosure find use in experimental animals, in veterinary application, and in the development of animal models, including, but not limited to, rodents including felines, mice, rats, hamsters, and primates. In some embodiments, the mammalian subject is a feline subject.

Subjects suitable for treatment in accordance with the methods described herein will vary and may include but are not limited to e.g., subjects suspected of having a viral infection or disease or condition mediated by a virus.

In some instances, subjects suitable for treatment in accordance with the methods described herein include subjects that do not have a viral infection or disease or condition but will be subjected to or otherwise exposed to conditions predicted to cause a viral infection or disease or condition. As such, in some instances, the methods described herein include preventing a viral infection or disease or condition in a subject that does not have a viral infection or disease or condition but is expected to be exposed to conditions that may cause a viral infection or disease or condition. In some embodiments, the treating further includes treating a condition resulting from a virus infection.

In one embodiment, the methods of treating a virus infection described herein relate to the treatment of a virus that is a coronavirus infection. The term “Coronavirus (CoV)” as used herein refers to viruses in the family Coronaviridae. In humans, CoV causes respiratory infections, which are typically mild but can be lethal in rare forms such as SARS (severe acute respiratory syndrome)-CoV, MERS (Middle East Respiratory Syndrome)-CoV, and COVID-19. Other exemplary human CoV include CoV 229E, CoV NL63, CoV OC43, CoV HKU1, and CoV HKU20. The envelope of CoV carries three glycoproteins: S-spike protein: receptor binding, cell fusion, major antigen; E-Envelope protein: small, envelope-associated protein; and M Membrane protein: transmembrane-budding & envelope formation. In a few types of CoV, there is a fourth glycoprotein: HE-heamagglutinin-esterase. In some embodiments, the virus infection may include, but are not limited to, a severe acute respiratory syndrome coronavirus 2 (SARS-CoV-2), severe acute respiratory syndrome coronavirus (SARS-CoV), Middle East respiratory syndrome-related coronavirus (MERS-CoV), Human coronavirus HKU1 (HCoV-HKU1), Human coronavirus OC43 (HCoV-OC43), Human coronavirus NL63 (HCoV-NL63), or Human coronavirus 229E (HCoV-229E) infection. In one embodiment, the virus infection is a SARS-CoV2 infection.

In one embodiment, the method further includes selecting a subject with a virus infection. In one embodiment, the selected subject has SARS, MERS, or COVID-19. The selected subject may, in some embodiments, have mild, moderate, or severe symptoms or exhibits no symptoms associated with SARS, MERS, or COVID-19.

In one embodiment, the virus infection is a feline coronavirus. The feline coronavirus may in some embodiments, be, for example, a feline enteric coronavirus (FECV) or feline infectious peritonitis virus (FIPV). In one embodiment, the virus infection is a canine coronavirus. In one embodiment, the virus infection is a type 2 canine or feline coronavirus. An example of feline infectious peritonitis virus strain is WSU 79-1146. An example of canine coronavirus strain is 1-71 (ATCC CRL-1542).

In the context of using compositions of the present disclosure treat a viral infection, disease, or condition, the amount is adequate to inhibit a viral infection, disease, or condition are capable of achieving a reduction in a number of symptoms, a decrease in the severity of at least one symptom, or a delay in the further progression of at least one symptom, or even a total alleviation of the infection.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. In some embodiments, the method includes repeated administering. Other therapeutic regimens or agents can be used in conjunction with the methods and compounds of the present disclosure. Adjustment and manipulation of established dosages (e.g., frequency and duration) are well within the ability of those skilled in the art. General guidance can be found, for example, in the publications of the International Conference on Harmonization and in REMINGTON'S PHARMACEUTICAL SCIENCES (Mack Publishing Company 1990), which is hereby incorporated by reference in its entirety.

Therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosage regimens can be adjusted to provide an optimum prophylactic or therapeutic response. The dose administered to a subject, in the context of the present disclosure should be sufficient to affect a beneficial therapeutic response in the patient over time. The size of the dose also will be determined by the existence, nature, and extent of any adverse side-effects. A therapeutically effective amount is also one in which any toxic or detrimental side effects of the compound and/or other biologically active agent is outweighed in clinical terms by therapeutically beneficial effects. A non-limiting range for a therapeutically effective amount of a compound and/or other biologically active agent within the methods and formulations of the disclosure is about 0.01 mg/kg body weight to about 50 mg/kg body weight. For example, the compound in some embodiments may be administered in a concentration of between about 0.01 mg/kg to about 5 mg/kg body weight, between about 0.05 mg/kg to about 50 mg/kg body weight, between about 1.0 mg/kg to about 50 mg/kg body weight, between about 1.5 mg/kg to about 50 mg/kg body weight, between about between about 2.0 mg/kg to about 50 mg/kg body weight, between about 2.5 mg/kg to about 50 mg/kg body weight, about 0.2 mg/kg to about 50 mg/kg body weight, or any amount between 0.1 mg/kg body weight and about 50 mg/kg body weight. In other embodiments, the compound may be administered in a concentration of between about 0.05 mg/kg to about 25 mg/kg body weight, between about 1.0 mg/kg to about 25 mg/kg body weight, between about 1.5 mg/kg to about 25 mg/kg body weight, between about between about 2.0 mg/kg to about 25 mg/kg body weight, between about 2.5 mg/kg to about 25 mg/kg body weight, about 0.2 mg/kg to about 25 mg/kg body weight, or any amount between 0.1 mg/kg body weight and about 25 mg/kg body weight.

Dosage can be varied by the attending clinician to maintain a desired concentration at a target site (for example, the lungs or systemic circulation). Higher or lower concentrations can be selected based on the mode of delivery, for example, trans-epidermal, rectal, oral, pulmonary, intraosseous, or intranasal delivery versus intravenous or subcutaneous or intramuscular delivery. Dosage can also be adjusted based on the release rate of the administered formulation, for example, of an intrapulmonary spray versus powder, sustained release oral versus injected particulate or transdermal delivery formulations, and so forth.

The amount of active ingredient that may be combined with the carrier materials to produce a single dosage form will vary depending upon the host treated and the particular mode of administration. For example, a formulation intended for the oral administration of humans may vary from about 0.1% to about 95% of the total composition. Dosage unit forms may contain between from about 0.1 mg to about 500 mg of active ingredient.

Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

The term “administering” means oral administration, administration as a suppository, topical contact, intravenous, parenteral, intraperitoneal, intramuscular, intralesional, intrathecal, intracranial, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

In certain embodiments, the method may include administering the compound in a time release formulation, for example in a composition which includes a slow-release polymer. These compositions can be prepared with vehicles that will protect against rapid release, for example a controlled release vehicle such as a polymer, microencapsulated delivery system or bio-adhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent, and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

The methods of the present disclosure and compounds described herein can be administered, e.g., by intravenous injection, intramuscular injection, subcutaneous injection, intraperitoneal injection, topical, sublingual, intraarticular (in the joints), intradermal, buccal, ophthalmic (including intraocular), intranasally (including using a cannula), or by other routes. The methods of the present disclosure and the compounds described herein (e.g., formulae I and/or II) can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g., WO 97/11682, which is hereby incorporated by reference in its entirety) via a liposomal formulation (see, e.g., European Patent No. 736299, WO 99/59550, and WO 97/13500, which are hereby incorporated by reference in their entirety), via formulations described in WO 03/094886, which is hereby incorporated by reference in its entirety, or in some other form. The compounds of the present disclosure can also be administered transdermally (i.e., via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al., Nature Reviews Drug Discovery 3:115 (2004), which is hereby incorporated by reference in its entirety). The compounds can be administered locally, for example, at the site of injury to an injured blood vessel. The compounds can be coated on a stent. The compounds can be administered using high-velocity transdermal particle injection techniques using the hydrogel particle formulation described in U.S. Patent Publication No. 20020061336, which is hereby incorporated by reference in its entirety. Additional particle formulations are described in WO 00/45792, WO 00/53160, and WO 02/19989, which are hereby incorporated by reference in their entirety. An example of a transdermal formulation containing plaster and the absorption promoter dimethylisosorbide can be found in WO 89/04179, which is hereby incorporated by reference in its entirety. WO 96/11705, which is hereby incorporated by reference in its entirety, provides formulations suitable for transdermal administration.

In some embodiments, the methods are carried out, or the compositions of the disclosure can be delivered, by the use of liposomes which fuse with the cellular membrane or are endocytosed, i.e., by employing receptor ligands attached to the liposome, which bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo (see, e.g., Al-Muhammed, J. Microencapsul. 13:293-306 (1996); Chonn, Curr. Opin. Biotechnol. 6:698-708 (1995); and Ostro, Am. J. Hosp. Pharm. 46:1576-1587 (1989), which are hereby incorporated by reference in their entirety). The methods of the present disclosure and compounds described herein can also be delivered as nanoparticles.

The methods and/or compositions of the present disclosure may additionally include components to provide sustained release and/or comfort. Such components include high molecular weight, anionic mucomimetic polymers, gelling polysaccharides, and finely divided drug carrier substrates. These components are discussed in greater detail in U.S. Pat. Nos. 4,911,920; 5,403,841; 5,212,162; and 4,861,760, which are hereby incorporated by reference in their entirety. The methods and/or pharmaceutical compositions of the disclosure can also be delivered as microspheres for slow release in the body. For example, microspheres can be administered via intradermal injection of drug-containing microspheres, which slowly release subcutaneously (see, e.g., Rao, J. Biomater Sci. Polym. Ed. 7:623-645 (1995), which is hereby incorporated by reference in its entirety; as biodegradable and injectable gel formulations (see, e.g., Gao Pharm. Res. 12:857-863 (1995), which is hereby incorporated by reference in its entirety); or, as microspheres for oral administration (see, e.g., Eyles, J. Pharm. Pharmacol. 49:669674, 1997, which is hereby incorporated by reference in its entirety).

Any pharmaceutically acceptable liquid carrier suitable for preparing solutions, suspensions, emulsions, syrups, and elixirs may be employed in the methods and compositions of the present disclosure. Compounds of the present disclosure may be dissolved or suspended in a pharmaceutically acceptable liquid carrier such as water, an organic solvent, or a pharmaceutically acceptable oil or fat, or a mixture thereof. The liquid composition may contain other suitable pharmaceutical additives such as solubilizers, emulsifiers, buffers, preservatives, sweeteners, flavoring agents, suspending agents, thickening agents, coloring agents, viscosity regulators, stabilizers, osmo-regulators, or the like. Examples of liquid carriers suitable for oral and parenteral administration include water (particularly containing additives as above, e.g., cellulose derivatives, preferably sodium carboxymethyl cellulose solution), alcohols (including monohydric alcohols and polyhydric alcohols, e.g., glycols) or their derivatives, or oils (e.g., fractionated coconut oil and arachis oil). For parenteral administration, the carrier may also be an oily ester such as ethyl oleate or isopropyl myristate.

The pharmaceutical agents of the present disclosure may be formulated for parenteral administration. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the pharmaceutical agents of the present disclosure systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents.

Intraperitoneal or intrathecal administration of the agents of the present disclosure can also be achieved using infusion pump devices such as those described by Medtronic, Northridge, CA. Such devices allow continuous infusion of desired compounds avoiding multiple injections and multiple manipulations.

In addition to the formulations described previously, the agents may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The nature of the carrier will depend on the particular mode of administration being employed. For instance, parenteral formulations may contain injectable fluids that include pharmaceutically and physiologically acceptable fluids such as water, physiological saline, balanced salt solutions, aqueous dextrose, glycerol, or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutically acceptable salts include, but are not limited to, amine salts, such as but not limited to, N, N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl- benzimidazole, diethylamine and other alkylamines, piperazine, and tris (hydroxymethyl) aminomethane; alkali metal salts, such as but not limited to, lithium, potassium, and sodium; alkali earth metal salts, such as but not limited to, barium, calcium, and magnesium; transition metal salts, such as but not limited to, zinc; and other metal salts, such as but not limited to, sodium hydrogen phosphate and disodium phosphate; and also including, but not limited to, salts of mineral acids, such as but not limited to, hydrochlorides and sulfates; and salts of organic acids, such as but not limited to, acetates, lactates, malates, tartrates, citrates, ascorbates, succinates, butyrates, valerates and fumarates.

Pharmaceutically acceptable esters include, but are not limited to, alkyl, alkenyl, alkynyl, aryl, heteroaryl, cycloalkyl and heterocyclyl esters of acidic groups, including, but not limited to, carboxylic acids, phosphoric acids, phosphinic acids, sulfonic acids, sulfinic acids, and boronic acids. Pharmaceutical acceptable enol ethers include, but are not limited to, derivatives of formula C═C(OR) where R is hydrogen, alkyl, alkenyl, alkynyl, aryl, heteroaryl, cycloalkyl, or heterocyclyl. Pharmaceutically acceptable enol esters include, but are not limited to, derivatives of formula C═C(OC(O)R), where R is hydrogen, alkyl, alkenyl, alkynyl, aryl, heteroaryl, cycloalkyl, or heterocyclyl. Pharmaceutical acceptable solvates and hydrates are complexes of a compound with one or more solvent or water molecules, or 1 to about 100, or 1 to about 10, or one to about 2, 3, or 4, solvent or water molecules.

Pharmaceutical compositions disclosed herein include those formed from pharmaceutically acceptable salts and/or solvates of the disclosed compounds. Pharmaceutically acceptable salts include those derived from pharmaceutically acceptable inorganic or organic bases and acids. Particular disclosed compounds possess at least one basic group that can form acid-base salts with acids. Examples of basic groups include, but are not limited to, amino and imino groups. Examples of inorganic acids that can form salts with such basic groups include, but are not limited to, mineral acids such as hydrochloric acid, hydrobromic acid, sulfuric acid, or phosphoric acid. Basic groups also can form salts with organic carboxylic acids, sulfonic acids, sulfa acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with a-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid. In particular, suitable salts include those derived from alkali metals such as potassium and sodium, alkaline earth metals such as calcium and magnesium, among numerous other acids well known in the pharmaceutical art. Descriptions of suitable pharmaceutically acceptable salts can be found in Handbook of Pharmaceutical Salts, Properties, Selection and Use, Wiley VCH (2002), which is hereby incorporated by reference in its entirety. When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977), which is hereby incorporated by reference in its entirety.

Certain compounds include at least one acidic group that can form an acid-base salt with an inorganic or organic base. Examples of salts formed from inorganic bases include salts of the presently disclosed compounds with alkali metals such as potassium and sodium, alkaline earth metals, including calcium and magnesium and the like. Similarly, salts of acidic compounds with an organic base, such as an amine (as used herein terms that refer to amines should be understood to include their conjugate acids unless the context clearly indicates that the free amine is intended) are contemplated, including salts formed with basic amino acids, aliphatic amines, heterocyclic amines, aromatic amines, pyridines, guanidines and amidines. Of the aliphatic amines, the acyclic aliphatic amines, and cyclic and acyclic di- and tri-alkyl amines are particularly suitable for use in the compounds. In addition, quaternary ammonium counterions can be used.

Particular examples of suitable amine bases (and their corresponding ammonium ions) for use in the present compounds include, without limitation, pyridine, N,N-dimethylaminopyridine, diazabicyclononane, diazabicycloundecene, N-methyl-N-ethylamine, diethylamine, triethylamine, diisopropylethylamine, mono-, bis- or tris-(2-hydroxyethyl) amine, 2-hydroxy-tert-butylamine, tris(hydroxymethyl) methylamine, N,N-dimethyl-N-(2-hydroxyethyl)amine, tri-(2-hydroxyethyl)amine and N-methyl-D-glucamine. For additional examples of “pharmacologically acceptable salts,” see Berge et al., J. Pharm. Sci. 66:1 (1977), which is hereby incorporated by reference in its entirety. Compounds disclosed herein can be crystallized and can be provided in a single crystalline form or as a combination of different crystal polymorphs. As such, the compounds can be provided in one or more physical form, such as different crystal forms, crystalline, liquid crystalline or non-crystalline (amorphous) forms.

Such different physical forms of the compounds can be prepared using, for example different solvents or different mixtures of solvents for recrystallization. Alternatively, or additionally, different polymorphs can be prepared, for example, by performing recrystallizations at different temperatures and/or by altering cooling rates during recrystallization. The presence of polymorphs can be determined by X-ray crystallography, or in some cases by another spectroscopic technique, such as solid phase NMR spectroscopy, TR spectroscopy, or differential scanning calorimetry.

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like. Pharmaceutical compositions for administering the compound can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

To formulate the pharmaceutical compositions, the compound of formula (I) and/or formula (II) can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80 or Miglyol 812), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel) or Freund's adjuvant, among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The compound of formula (I) and/or formula (II) can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively, or additionally, synthetic fatty acid esters such as polyglycerin fatty acid esters, sucrose fatty acid esters and the like can be employed as vehicles. Hydrophilic polymers and other vehicles can be used alone or in combination and enhanced structural integrity can be imparted to the vehicle by partial crystallization, ionic bonding, cross-linking and the like. The vehicle can be provided in a variety of forms, including fluid or viscous solutions, gels, pastes, powders, microspheres, and films for direct application to a mucosal surface.

The methods of the present disclosure may further include administering one or more additional therapeutic agents in conjunction with said administering the compound of formula (I) or the compound of formula (II), depending upon the nature of the viral infection that is being treated. Such additional active agents include but are not limited to an antiviral compound, an immune modulator, a convalescent plasma, or a combination thereof. Examples of additional therapeutic agents that may be used in the methods of the disclosure include but are not limited to GS-441524, Diosmin, Hesperidin, MK-3207, Venetoclax, Dihydroergocristine, Bolazine, R428, Ditercalinium, Etoposide, Teniposide, UK-432097, Irinotecan, Lumacaftor, Velpatasvir, Eluxadoline, Ledipasvir, Lopinavir/Ritonavir Ribavirin, Alferon and prednisone. Other additional agents that may be useful in the methods of the present disclosure include dexamethasone, azithromycin, and remdesivir, as well as boceprevir, umifenovir, and favipiravir. Other additional agents that may be useful include for example those described in U.S. Pat. No. 11,351,114 which is hereby incorporated by reference in its entirety. Co-administration is meant to include simultaneous or sequential administration of the formula (I) and/or formula (II) of the present disclosure individually or in combination with another active agent (more than one compound or agent). Thus, the methods and/or compositions of the disclosure can also be combined, when desired, with other active substances (e.g., to reduce metabolic degradation).

Another aspect of the present disclosure relates to a method of inhibiting viral replication by contacting one or more cells infected with a virus with an effective amount of a compound of either formula (I), formula (II), or a pharmaceutically acceptable salt thereof. The various embodiments of formula (I) and formula (II) are in accordance with the previously described aspect.

The effective amount of a compound of formula (I) or formula (II) can be adjusted to provide an optimum inhibition of viral replication. The effective amount, in the context of the present aspect should be sufficient to cause an inhibition of viral replication in one or more cells. A non-limiting range for an effective amount of a compound and/or other biologically active agent within the methods and formulations of the disclosure is about 0.01 μM to about 50 μM. For example, the compound in some embodiments may be provided in a concentration of between about 0.01 μM to about 5 μM, between about 0.05 μM to about 50 μM, between about 1.0 μM to about 50 μM, between about 1.5 μM to about 50 μM, between about between about 2.0 μM to about 50 μM, between about 2.5 μM to about 50 μM, about 0.2 μM to about 50 μM, or any amount between 0.1 μM and about 50 μM. In other embodiments, the compound may be administered in a concentration of between about 0.05 μM to about 25 M, between about 1.0 μM to about 25 μM, between about 1.5 μM to about 25 μM, between about between about 2.0 μM to about 25 μM, between about 2.5 μM to about 25 μM, about 0.2 μM to about 25 μM, or any amount between 0.1 μM and about 25 μM.

Another aspect of the present disclosure relates to a composition comprising the compound of formula (I) or a pharmaceutically acceptable salt thereof. The various embodiments of formula (I) are in accordance with the previously described aspects.

The compounds of the present disclosure (or pharmaceutically acceptable salts, esters, enol ethers, enol esters, solvates, hydrates, or prodrugs thereof) can optionally be modified to include a tag. A “tag” as used herein includes any labeling moiety that facilitates the detection, quantitation, isolation, and/or purification of a compound (i.e., a compound of the present disclosure). Methods for modifying small molecules to include tags are well known in the art. For example, click chemistry (see, e.g., U.S. Pat. No. 7,375,234 to Sharpless et al., which is hereby incorporated by reference in its entirety) may be used to attach a tag to a compound.

Suitable tags include purification tags, radioactive or fluorescent labels, enzymatic tags, prosthetic groups, luminescent materials, bioluminescent materials, positron emitting metals, nonradioactive paramagnetic metal ions, and any other signal suitable for detection and/or measurement by radiometric, colorimetric, fluorometric, size-separation, or precipitation means, or other means known in the art.

Purification tags, such as maltose-binding protein (MBP-), poly-histidine (His₆-), or a glutathione-S-transferase (GST-), can assist in compound purification or separation but can later be removed, i.e., cleaved from the compound following recovery. Protease-specific cleavage sites can be used to facilitate the removal of the purification tag. The desired product can be purified further to remove the cleaved purification tags.

Other suitable tags include radioactive labels, such as, ¹²⁵I, ¹²³I, ¹³¹I, ¹¹¹In, ¹¹²In, ¹¹³In, ¹¹⁵In, ⁹⁹Tc, ²¹³Bi, ¹⁴C, ⁵¹Cr, ¹⁵³Gd, ¹⁵⁹Gd, ⁶⁸Ga, ⁶⁷Ga, ⁶⁸Ge, ¹⁶⁶Ho, ¹⁴⁰La, ¹⁷⁷Lu, ⁵⁴Mn, ⁹⁹Mo, ¹⁰³Pd, ³²P, ¹⁴²Pr, ¹⁴⁹Pm, ¹⁸⁶Re, ¹⁸⁸Re, ¹⁰⁵Rh, ⁹⁷Ru, ¹⁵³Sm, ⁴⁷Sc, ⁷⁵Se, ⁸⁵Sr, ³⁵S, ²⁰¹Ti, ¹¹³Sn, ¹¹⁷Sn, ³H, ¹³³Xe, ¹⁶⁹Yb, ¹⁷⁵Yb, ⁹⁰Y, and ⁶⁵Zn. Methods of radiolabeling compounds are known in the art and described in U.S. Pat. No. 5,830,431 to Srinivasan et al., which is hereby incorporated by reference in its entirety. Radioactivity is detected and quantified using a scintillation counter or autoradiography. Further examples include positron emitting metals using various positron emission tomographies, and nonradioactive paramagnetic metal ions.

Alternatively, the compound can be conjugated to a fluorescent tag. Suitable fluorescent tags include, without limitation, chelates (europium chelates), fluorescein and its derivatives, rhodamine, and its derivatives, dansyl, Lissamine, phycoerythrin, Texas Red, and umbelliferone. The fluorescent labels can be conjugated to the compounds using techniques disclosed in CURRENT PROTOCOLS IN IMMUNOLOGY (Coligen et al. eds., 1991), which is hereby incorporated by reference in its entirety. Fluorescence can be detected and quantified using a fluorometer.

Enzymatic tags generally catalyze a chemical alteration of a chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Examples of suitable enzymatic tags include luciferases (e.g., firefly luciferase and bacterial luciferase; see e.g., U.S. Pat. No. 4,737,456 to Weng et al., which is hereby incorporated by reference in its entirety), luciferin, 2,3-dihydrophthalazinediones, malate dehydrogenase, urease, peroxidases (e.g., horseradish peroxidase), alkaline phosphatase, 0-galactosidase, glucoamylase, lysozyme, saccharide oxidases (e.g., glucose oxidase, galactose oxidase, and glucose-6-phosphate dehydrogenase), heterocyclic oxidases (e.g., uricase and xanthine oxidase), lactoperoxidase, microperoxidase, and the like. Techniques for conjugating enzymes to proteins and peptides are described in O'Sullivan et al., Methods for the Preparation of Enzyme-Antibody Conjugates for Use in Enzyme Immunoassay, in METHODS IN ENZYMOLOGY 147-66 (Langone et al. eds., 1981), which is hereby incorporated by reference in its entirety.

Prosthetic group complexes include, but are not limited to, streptavidin/biotin and avidin/biotin. Alternatively, the compound can be conjugated to a luminescent or bioluminescent material including, but not limited to, luminol, luciferase, luciferin, and aequorin.

The compounds of the present disclosure (or pharmaceutically acceptable salts, esters, enol ethers, enol esters, solvates, hydrates, or prodrugs thereof) can optionally be modified to include an attachment to a solid surface, such as a fibrous test strip, a column, a multi-well microliter plate, a test tube, or beads. Methods for attaching small molecules to such surfaces, including covalent attachment (for example via click chemistry, as described supra) as well as non-covalent attachment through the use of antibody-antigen partners, complementary nucleic acids, etc., are well known in the art.

The present disclosure also includes kits, packages, and multi-container units containing the herein described compositions, active ingredients, and/or means for administering the same for use in the prevention and treatment of diseases and other conditions in mammalian subjects.

In one embodiment, these kits include a container or formulation that contains one or more of the compounds described herein. In one example, this component is formulated in a pharmaceutical preparation for delivery to a subject. The compound is optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the pharmaceutical agent packaged therewith can be used.

### EXAMPLES

The following examples are intended to exemplify the practice of embodiments of the disclosure but are by no means intended to limit the scope thereof.

### Materials and Methods for Examples 1-6

### Materials

Coronaviruses—SARS-CoV-2 WA-1 (ATCC BEI Resources NR-52281); HCoV-OC43 (Betacoronavirus 1) (ATCC VR-1558); HCoV-229E (Human coronavirus 229E) (ATCC VR-740).

CellLines—HCT8 (human colorectal carcinoma cell line initiated from an adult male, ATCC CCL-244); HBEC3-KT (Primary human bronchial epithelial cells, ATCC CRL-4051); MRC5 (normal human fetal lung fibroblast cells, ATCC CCL-171); VeroE6 (ATCC CL 1586.

Antibodies—GLS antibody was raised against the sequence-KLDPRREGGDQRHS (SEQ ID NO: 1); GLS2 antibody was obtained from ProSci (Cat. #6217); Phospho-c-Jun (Ser73) antibody (Cat. #9164), Phospho-c-Jun (Ser63) II antibody (Cat. #9261), c-Jun (60A8) Rabbit mAb antibody (Cat. #9165), and Vinculin (E1E9V) XP Rabbit mAb antibody (Cat. #13901) were all obtained from Cell Signaling Technology. Anti-Coronavirus nucleoprotein of OC43, clone 542-7D antibody (Cat. #MAB9013) was from Millipore Sigma; SARS-CoV-2/2019-nCoV Spike/S2 Antibody (Cat. #40590-T62) was from Sino Biological; Goat anti-Rabbit IgG (H+L) Highly Cross-Adsorbed Secondary Antibody, Alexa Fluor™ 568 (Cat. #A-11036); Goad anti-Mouse IgG (H+L) Cross-Adsorbed Secondary Antibody, Alexa Fluor™ 488 (Cat. #A-11001; NucleoSpin RNA Virus kit (Takara Bio, REF #740956) was from Takara Bio; Direct-zol RNA MicroPrep kit (Cat. #R2062) was from ZYMO Research; Anti-rabbit second antibody (Cat. T2767) was from Invitrogen.

mRNA Silencers—pLKO.1-puro shRNA control plasmid (Cat. #SHC002), pLKO.1-TRCN0000051135 targeting GLS (Clone ID: NM_014905.2-1441s1c1), pLKO.1-TRCN0000298987 target GLS (Clone ID: 014905.3-1475s21c1), Silencer Select Negative Control No. 1 siRNA (Cat #4390843) were from Sigma-Aldrich; Silencer Select GLS siRNA (ID #s5838), and Silencer Select GLS2 siRNA (ID #5840) were from Invitrogen.

Inhibitors—Compound B and Compound C were obtained; Compound D (Cat. SML2472) was from Sigma-Aldrich; Compound A was from ChemBridge Corporation; JNK inhibitor SP600125 was from Sigma-Aldrich (Cat #A10860); c-Myc inhibitor 10058-F4 was from Sigma-Aldrich (Cat #A13725).

RNAs isolation kits—Direct-zol RNA MicroPrep was from ZYMO Research (cat. R2062); NucleoSpin RNA Virus was from Takara (cat. 740956)

### Qpcr Primers and Kit

SARS-CoV-2-spike-F
(SEQ ID NO: 2)
(CAACTGAAATCTATCAGGCCG)
SARS-CoV-2-spike-R
(SEQ ID NO: 3)
(ACCAACACCATTAGTGGGTTG)
HCoV-OC43-F
(SEQ ID NO: 4)
(CCCAAGCAAACTGCTACCTCTCAG)
HCoV-OC43-R
(SEQ ID NO: 5)
(CCCAAGCAAACTGCTACCTCTCAG)
HCoV-229E-F
(SEQ ID NO: 6)
(TCTGCCAAGAGTCTTGCTCG)
HCoV-229E-R
(SEQ ID NO: 7)
(TCTGCCAAGAGTCTTGCTCG)
GLS-F
(SEQ ID NO: 8)
(TGTCACGATCTTGTTTCTCTGTG)
GLS-R
(SEQ ID NO: 9)
(TCATAGTCCAATGGTCCAAAG)
GLS2-F
(SEQ ID NO: 10)
(GCCTGGGTGATTTGCTCTTTT)
GLS2-R
(SEQ ID NO: 11)
(CCTTTAGTGCAGTGGTGAACTT)
actin-F
(SEQ ID NO: 12)
(CATCGAGCACGGCATCGTCA)
actin-R
(SEQ ID NO: 13)
(TAGCACAGCCTGGATAGCAAC)

iTaq™ Universal SYBR® Green One-Step Kit was from Bio-Red (cat. #1725151).

### Methods

SARS-CoV-2 propagation and infection—Studies of SARS-CoV-2 were performed in a BSL3 lab. VerE6 cells were grown in 15-cm plates until 80-90% confluent. SARS-CoV-2 virus dilution was prepared in 7 ml of DMEM 2% FBS (MOI 0.05) per plate. After washing with PBS two times, virus dilution was added to the cell plates for 2 hours at 33° C. in 5% CO₂ with continuous shaking. End of adsorption by adding 10 ml DMEM 2% FBS medium and incubation for 4-6 days or until the cytopathic effect (CPE) progressed through 80% of the cells. The virus medium was collected and centrifuged at 1200 rpm for 10 mins. The supernatant containing the virus was aliquot and stored at −80° C. and in liquid Nitrogen. The virus titer was determined by plaque assay and qPCR.

VeroE6 cells were grown in 10 cm plates until 70% confluent, pre-treated with inhibitors for 3 hours, and then infected with SARS-CoV-2 (MOI 0.01) for 1 hour at 33° C. 5% CO₂ with shaking. The plate media was changed to the growth medium with or without inhibitors and continued incubated for 23 hours at 37° C. and 5% CO₂. The medium and cells were collected, and the viruses were inactivated for qPCR and Western blot assay, respectively.

HCoV-OC43 propagation and infection—The HCoV-OC43 virus propagation followed the ATCC protocol. HCT8 cells were grown in 15-cm plates until 80-90% confluent. Virus dilution was prepared in 7 ml of RPMI medium containing 2% HS (MOI 0.05). The monolayer cell plates were washed two times with PBS. Virus dilution was adsorbed by cells for 2 hours at 33° C., 5% CO₂ incubator while shaking continuously. End adsorption by adding 10 ml RPMI with 2% HS medium and continue incubation for 4-6 days at 33° C., 5% CO₂ incubator with shaking. The virus medium was collected when the CPE progressed through 80% of the monolayer. The virus medium was centrifuged at 1200 rpm for 10 mins and aliquots of the supernatant containing the virus were stored at −80° C. or in a liquid nitrogen tank for the long term. The virus titer was determined by plaque assay and qPCR.

HCT8 cells or HBEC cells were grown in 10 cm plates until 70-80% confluent at 37° C., 5% CO₂, and pretreated with inhibitors for 3 hours. HCoVOC43 virus (MOI 0.01, RPMI 2% HS) was infected for 1 hour at 33° C. 5% CO₂ with shacking. The different conditions of the growth medium of HCT8 or HBEC were changed as designed and the cell plates were continued to be incubated at 37° C. 5% CO₂ incubator for 23 hours. The cells and media were collected for Western blot and qPCR, respectively.

HCoV-229E virus propagation and infection—Following the ATCC protocol for virus propagation. MRC5 (EMEM 10% FBS) cells were grown in the 15 cm plates, until 80-90% confluent. The virus dilution was prepared in 7 ml of EMEM with 2% FBS medium, MOI 0.05. The cells were adsorbed with virus dilution at 35° C. 5% CO₂ shaking for 2 hours. Ten ml of EMEM with 2% FBS medium was added to the plates and incubated for 4-6 days until CPE progressed to 80%. The virus media were collected and centrifuged at 1200 rpm for 10 mins. The supernatant containing viruses was aliquoted, and the virus tubes were stored at −80° C. The virus titer was determined by qPCR and plaque assays.

MRC5 and HBEC cells were grown for 70-80% confluent at 37° C. 5% CO2, were pretreated with the inhibitors for 3 hours, then were infected with the virus (MOI 0.01, EMEM 2% FBS) dilution for 1 hour at 35° C., 5% CO₂ with shaking. The cell media were changed to the appropriate conditions of growth media and the cells were incubated for 23 hours at 37° C., 5% CO₂ incubator. The cells and media were collected for further analysis.

Western blot analysis—Western blot analyses were performed as described (Greene et al., “SIRT5 Stabilizes Mitochondrial Glutaminase and Supports Breast Cancer Tumorigenesis,” Proc. Natl. Acad. Sci. USA 116(52):26625-26632 (2019), which is hereby incorporated by reference in its entirety). Briefly, the cells were collected from different conditions and prepared with lysis buffer. Protein concentration was determined by Bradford assay (Bio-Rad), and lysate proteins were denatured by boiling for 5 min. Lysate proteins were resolved on Tris-glycine protein gels (Life Technologies) and then transferred to PVDF membranes (PerkinElmer). Membranes were blocked with milk (5%) or BSA(10%) in TBST for at least one hour and incubated with primary antibodies dilutions in TBST overnight at 4° C. Horseradish-peroxidase-conjugated secondary antibodies were used to detect primary antibodies, followed by imaging using Western Lighting Plus-ECL (PerkinElmer) and the film was used to detect the antibodies binds.

Immunofluorescence staining—The HBECs or HCT8 cells were grown in the 4-well slide chamber, and diluted HCoV-OC43 viruses (MOI 0.01) were adsorbed for 1 hour at 33° C., 5% CO₂. The medium was changed to growth medium, and the cells were maintained at 37° C. 5% CO₂ for 22 hours. One drop of NucBlue Live Cell staining solution was added to each cell well for 1 hour. The cells were fixed with 3.7% formaldehyde for 20 minutes and washed with PBS (3×). GLS antibody (1:500) was added into the cell chamber at room temperature for 2 hours, the cells were washed with PBS (3×), and then HCoV-OC43 antibody (1:500) was added into the chamber for 2 hours at room temperature. The mix of Goat anti-rabbit second antibody, Alexa Fluor 568 (1:200) for GLS primary antibody and Goat anti-Mouse secondary antibody, Alexa Fluor 488 (1:200) for HCoV-OC43, was applied to the chamber and incubated at room temperature for 1 hour with rocking. The cells were washed with PBS (3×). The images were taken with KEYENCE BZ-X810 microscope.

RNA isolation from cell and medium—RNA was isolated either from the cells or from media with the kits Direct-zol RNA MicroPrep kit and NucleoSpin RNA virus kit, respectively.

Quantitative PCR (qPCR)—Total RNA was isolated from cells or media using qPCR isolation kits Direct-zol RNA MicroPrep from ZYMO Research and NucleoSpin RNA Virus from Takara, respectively. qPCR analysis was carried out using RNA as a template, specific primers and iTaa™ Universal SYBR© Green One-Step Kit (Bio-Rad). Reactions were performed using the real-time PCR system (7500 fast real-time PCR system, Applied Biosystems).

Metabolite Extraction—HBECs were grown in 6-well plates at 80% confluence (triplets for each condition), pretreated with Compound B (2.5 uM) or Compound D (0.5 uM) for 3 hours with non-treated wells as controls, and then infected with HCoV-OC43(MOI 0.1) for 1 hour at 33° C. 5% CO₂ with shaking, followed by media changed to the growth media with or without inhibitors and continued to incubate for 23 hours. The cell plates were placed on dry ice, and 1 ml of ice-cold extraction solution was added to each well of the cells with the cells remaining on dry ice for 10 minutes. The cells were scraped in the extraction solution, and the whole mixture of each condition was transferred to an Eppendorf tube. The tubes were incubated on dry ice for 1 hour and then spun down at 13000 rpm for 10 minutes. Supernatants (700 ul) from each sample were transferred to new tubes, and then samples were analyzed at the Cold Spring Harbor Metabolomic Facility.

Genetic Knockdowns using shRNA and siRNA—Knockdown of GLS expression in HBECs and HCT8 cells were achieved using short hairpin RNA (shRNA, MISSION RNAi system, Sigma-Aldrich). Lentivirus particles for each shRNA construct were generated using exponentially growing 293T cells (ATCC) as described previously (Greene et al., “SIRT5 Stabilizes Mitochondrial Glutaminase and Supports Breast Cancer Tumorigenesis,” Proc. Natl. Acad. Sci. USA 116(52):26625-26632 (2019) and Lukey et al., “Liver-Type Glutaminase GLS2 Is a Druggable Metabolic Node in Luminal-Subtype Breast Cancer,” Cell Rep. 29(1):76-88 (2019), both of which are hereby incorporated by reference in its entirety). Silencer Select pre-designed siRNAs (Invitrogen) targeting GLS and the control silencer were transfected into HBEC and HCT8 cells, using 60 mm dish, with 0.3 mL Opti-MEM(GIBCO) containing 100 nM of the appropriate siRNA to give the final concentration of 10 nM, along with 0.3 mL Opti-MEM containing 12 μl of Lipofectamine 2000 (Invitrogen), were incubated separately at room temperature for 5 mins. The two solutions were then combined and incubated for an additional 20 min, mixed with 2.4 mL culture medium, and added to cells. After 5 hours of incubation at 37° C., the transfection mixture was replaced with a fresh culture medium. For all the knockdowns, two independent shRNA or siRNA were used, along with the negative control shRNA or siRNA.

Virusplaque assay—A total of 2×10⁵ host cells were seeded in 6-well plates with culture medium and incubated for 24 hours at 37° C. The wells were washed with PBS, then the cells were infected with mock supernatant or different dilutions of virus (100p), in triplicate per condition. The plates were incubated at 37° C. for 4 hours and mixed gently every 15 mins. Sterile 4% oxoid agar with water as stock. 0.4% oxoid ager with culture medium was prepared and 2 mL was applied to each cell well after the medium was depleted. The cell plates were placed in a tissue culture hood for 15 min as the agar overlay turned solid, then incubated at 37° C. 5% CO₂ for 5-7 days. PFA (4%) was used to fix the cells for 30 min at room temperature, and then the ager was removed. Following immunostaining with virus antibody or Crystal violet staining, plaques were counted.

Statistical analyses—All differences were analyzed with one-way ANOVA with Bonferroni correction. All experiments were repeated independently at least three times.

### Example 1—Glutaminase expression is upregulated during Coronavirus replication

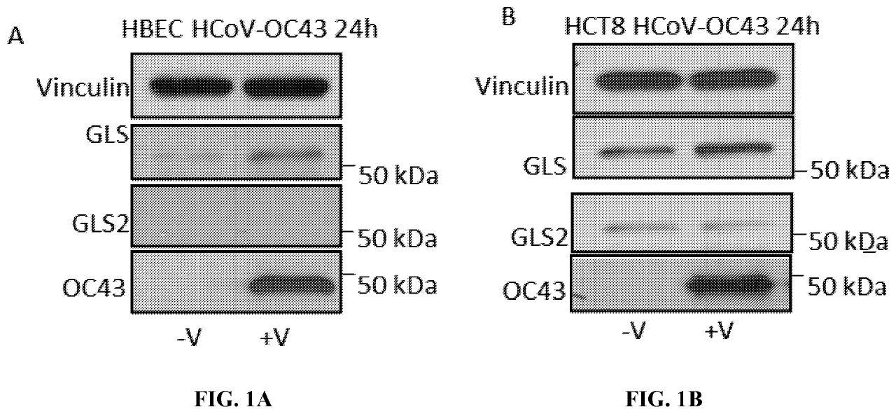

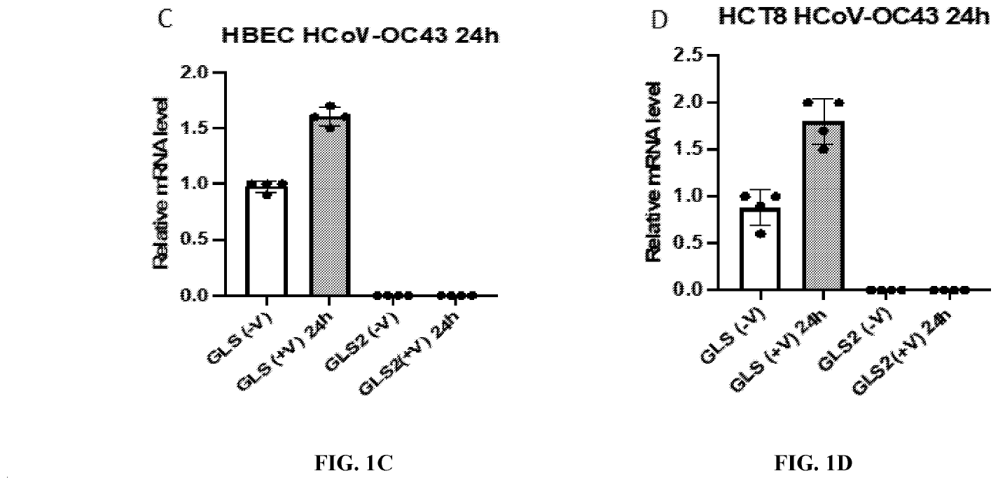

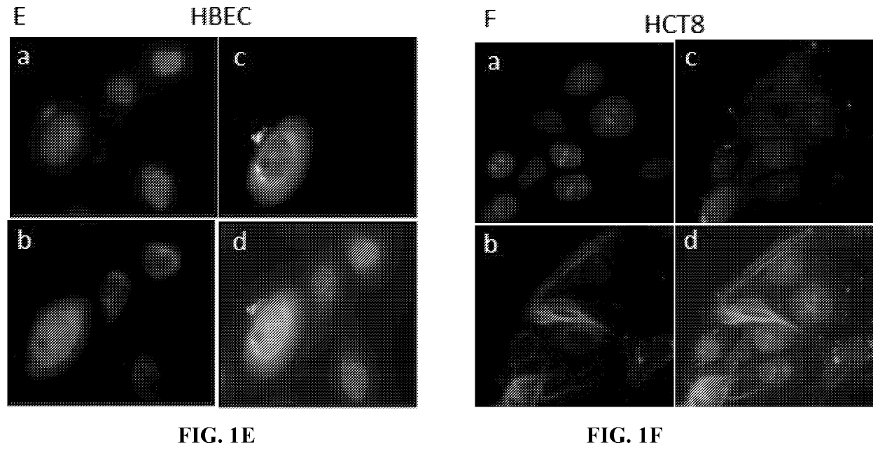

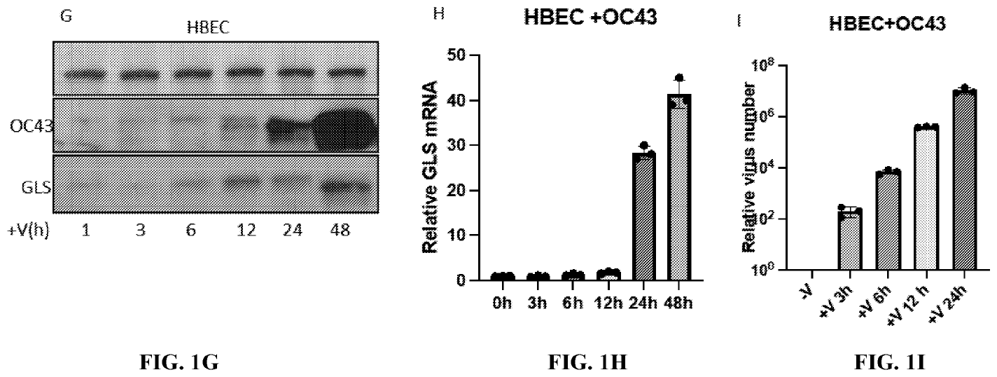

Glutamine metabolism has been suggested to be elevated and potentially contribute to the infection of host cells by various viruses. Mayer et al., “Hijacking the Supplies: Metabolism as a Novel Facet of Virus-host Interaction,” Front Immunol. 10(JULY):1-12 (2019) and Thai et al., “MYC-induced Reprogramming of Glutamine Catabolism Supports Optimal Virus Replication,” Nat. Commun. 6(1):8873 (2015), both of which are hereby incorporated by reference in their entirety. To ascertain whether this was the case for Coronavirus infection, it was determined whether glutaminase expression was upregulated when host cells were infected with a member of the Coronavirus family, HCoV-OC43. HBECs and HCT8 cells, when 80% confluent, were infected with HCoV-OC43 for 24 hours, and then the cells and their medium were collected. Western Blot analyses were performed using anti-HCoV-OC43 to detect the viral nucleoprotein OC43 as a read-out for viral replication, and anti-GLS and anti-GLS2 antibodies were used to detect the two forms of glutaminase expressed in the human epithelial cells (FIGS. 1A and 1i). Although GLS protein expression was relatively low in HBECs, it was increased significantly upon HCoV-OC43 infection. On the other hand, GLS2 protein expression was not detected either before or after virus infection, indicating that only GLS is potentially involved in Coronavirus HCoV-OC43 replication in HBECs. In HCT8 cells, GLS protein expression was modestly increased with virus infection, whereas GLS2 protein levels were not changed (FIG. 1B), although the basal levels of both GLS and GLS2 were higher in HCT8 cells compared to HBECs, most likely because the former represents a human colon cancer cell line. Quantitative PCR (qPCR) assays showed that the RNA transcript levels of GLS but not GLS2 were upregulated in virus infected HBECs and HCT8 cells (FIGS. 1C, 1D).

Immunofluorescence experiments were then carried out in HBEC and HCT8 cells to visualize GLS expression as a function of virus infection. Cells were infected with HCoV-OC43 as described above for 1 hour and then incubated with the culture medium for 23 hours. After fixation, the cells were incubated with antibodies specific for either GLS or HCoV-OC43 N protein, with the cell nuclei being visualized by NucBlue staining. Four HBECs shown in FIG. 1E-a (blue) exhibited different levels of GLS (FIG. 1E-b, red) and HCoV-OC43 expression (FIG. 1E-c, green). The merged images show that the virus-infected cells consistently expressed higher levels of GLS (FIG. 1E-d). The same was true upon HCoV-OC43 infection of HCT8 cells (FIG. 1F).

Next, a time course for HCoV-OC43 infection of HBECs was performed. The cells (70-80% confluent) were infected with HCoV-OC43 for 1 hour, with the medium from one plate of cells then being collected. The cells were cultured in standard HBEC medium, incubated at 370° C. and then collected, together with their medium, after 3, 6, 12, 24, and 48 hours. The expression levels of the HCoV-OC43 nucleoprotein OC43 and GLS were detected by Western Blot analyses (FIG. 1G), while qPCR was used to measure the total RNA transcript levels of GLS (FIG. 1H) and that of viral RNA in the media (FIG. 1I). Both OC43 and GLS expression levels showed significant increases between 12 and 48 hours of virus infection. The increase in GLS expression followed the time course for that of the virus protein (FIG. 1G), and the same was true for their RNA transcript levels (FIGS. 1H, 1I), indicating that the upregulation of GLS expression began with the onset of virus infection

### Example 2—GLS is Essential for Coronavirus Replication in HBECs

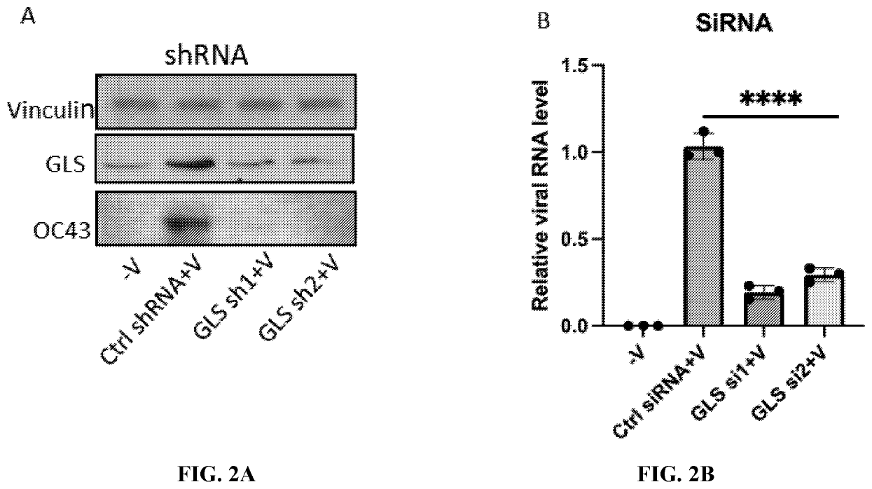

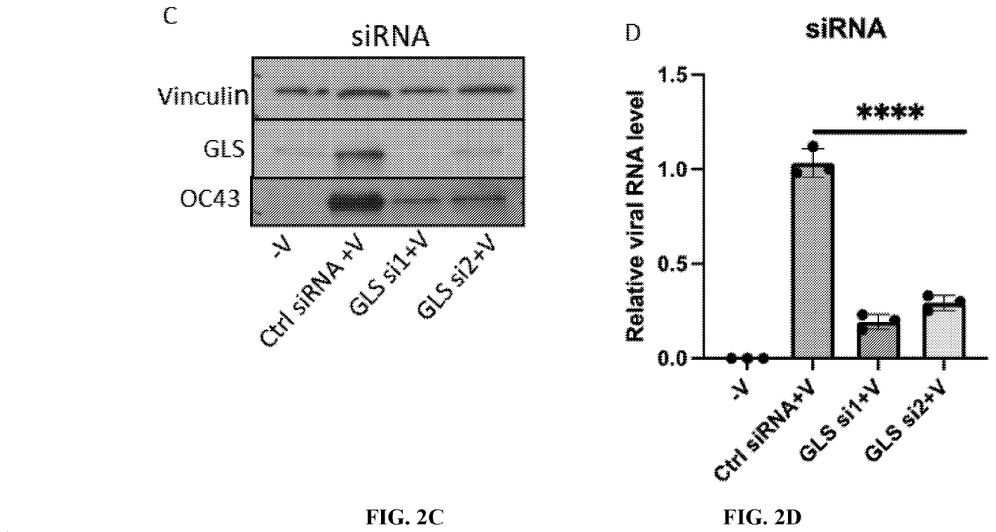

To determine whether GLS expression was necessary for Coronavirus infection, GLS was knocked down in HBECs, using two different shRNAs that target the enzyme. The cells were then infected with HCoV-OC43 for 24 hours, and cell lysates were analyzed by Western blot to determine GLS expression, while their medium was collected and analyzed by qPCR to detect total viral RNA. HCoV-OC43 replication was reduced significantly when GLS was depleted from the cells, as evidenced by the decrease in the expression of the viral protein OC43 (FIG. 2A) and viral RNA in the medium (FIG. 2B). Similar results were obtained when the GLS knock-down experiments were repeated by using two independent siRNAs targeting GLS followed by Western blot analyses to examine GLS and HCoV-OC43 levels, together with qPCR to assay viral RNA levels in the medium as a result of viral shedding (FIGS. 2C, 2D)

### Example 3—GLS Expression is Upregulated in HBECs by c-Jun During Virus Infection

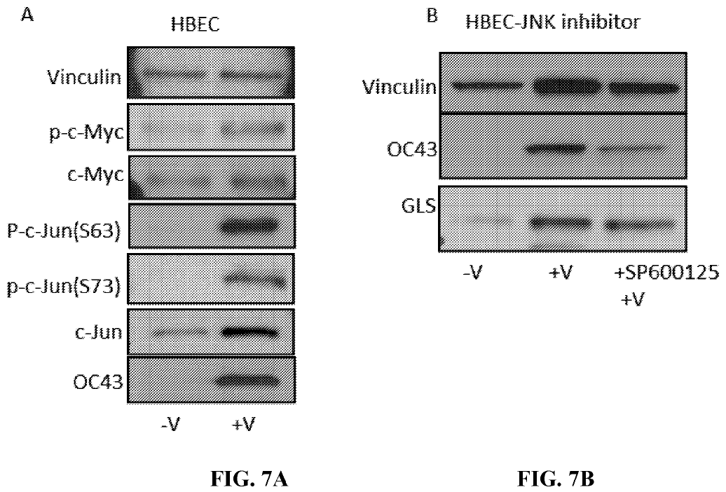

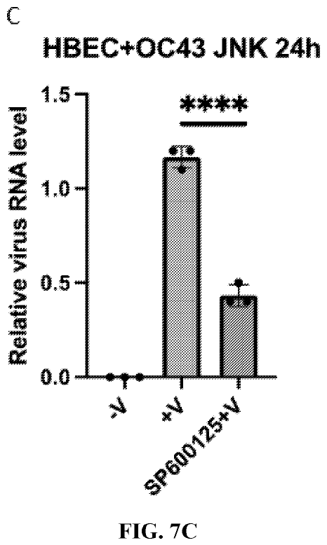

GLS expression in cancer cells has been reported to be upregulated either by c-Myc or c-Jun (Gao et al., “C-Myc Suppression of miR-23a/b Enhances Mitochondrial Glutaminase Expression and Glutamine Metabolism,” Nature 458(7239):762-765 (2009) and Lukey et al., “The Oncogenic Transcription Factor c-Jun Regulates Glutaminase Expression and Sensitizes Cells to Glutaminase-targeted Therapy,” Nat. Commun. 7:1-14 (2016), both of which are hereby incorporated by reference in their entirety). To further examine how GLS is upregulated upon virus infection, HBECs were infected with HCoV-OC43 for one hour followed by a 23-hour incubation. The cells were collected, and Western blot analyses were performed. Phospho-c-Myc, phospho-c-Jun(S63) and phospho-c-Jun(S73) were all observed to be increased after infection, as were the total protein expression levels of c-Myc and c-Jun (FIG. 7A). However, while inhibiting c-Myc did not cause a reduction in the expression levels of GLS in virus-infected host cells (not shown), blocking c-Jun activation using the small molecule inhibitor SP600125 reduced both GLS protein expression and the amount of viral RNA transcripts detected in the medium (FIG. 7B)

### Example 4—Glutaminase Inhibitors Block Coronavirus SARS-Co-V2 and HCov-OC43 Replication

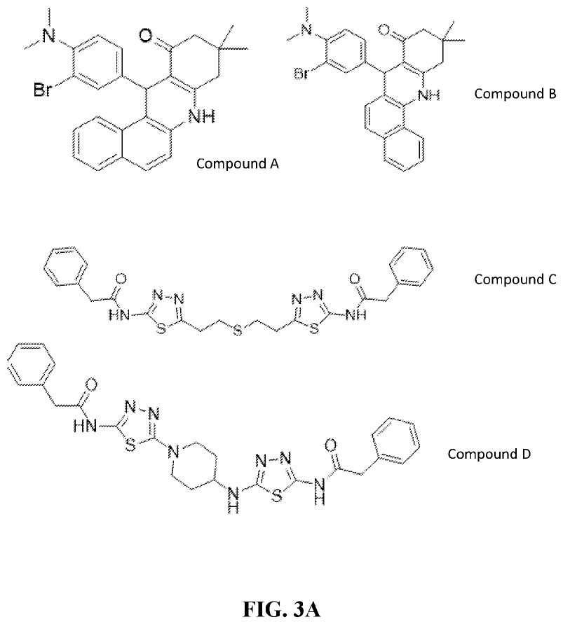

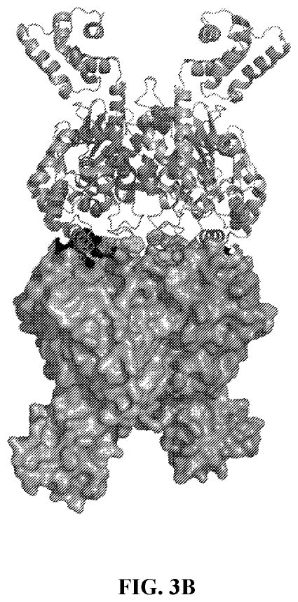

It was next examined whether small molecule allosteric inhibitors targeting the glutaminase enzymes could impact Coronavirus infection. Two types of allosteric glutaminase inhibitors have been developed and characterized, designated as the Compound A and Compound C class of molecules. McDermott et al., “Design and Evaluation of Novel Glutaminase Inhibitors,” Bioorg. Med. Chem. 24(8):1819-1839 (2016); Katt et al., “Dibenzophenanthridines as Inhibitors of Glutaminase C and Cancer Cell Proliferation,” Mol. Cancer Ther. 11(6):1269-1278 (2012); Yuan et al., “Broad-spectrum Antiviral Target,” Nat. Commun. 10(1):120 (2019); and Stalnecker et al., “Mechanism by Which a Recently Discovered Allosteric Inhibitor Blocks Glutamine Metabolism in Transformed Cells,” Proc. Natl. Acad. Sci. USA 112(2):394-399 (2014), all of which are hereby incorporated by reference in their entirety. X-ray crystal structures have shown that Compound C, its more potent analogs CB839 and our more newly developed inhibitor Compound D bind in the interface where two dimers of GLS come together to form a tetramer. Based on docking analyses, Compound A and the more potent analog Compound B have been suggested to nestle into a cove that forms between two monomers within a GLS dimer (FIG. 3B) (Stalnecker et al., “Mechanism by Which a Recently Discovered Allosteric Inhibitor Blocks Glutamine Metabolism in Transformed Cells,” Proc. Natl. Acad. Sci. USA 112(2):394-399 (2014), which is hereby incorporated by reference in its entirety). The Compound C group of GLS inhibitors traps the enzyme in an inactive tetrameric state while the Compound A group of compounds initially bind to the monomeric form of the enzyme and stabilize an inactive dimer. Seven Coronaviruses can infect humans. In these experiments, three of them were examined with their host cells: HCoV229E, which belongs to the alpha sub-class of the Coronavirus family, and SARS-CoV-2 and HCoV-OC43 from the beta-subclass (Table 1).

TABLE 1
Human Coronaviruses and Host Cells

| HCoronavirus line | Sub-type | Used | Host Cell |
|---|---|---|---|
| Hcov-229E | Alpha | Yes | MRC5 |
| HCoV-NL63 | Alpha | No | |
| HCoV-OC43 | Beta | Yes | HCT8 |
| SARS-CoV-2 | Beta | Yes | VeroE6 |
| HCoV-HUK1 | Beta | No | |
| MERS-CoV | Beta | No | |
| SARS-CoV | Beta | No | |

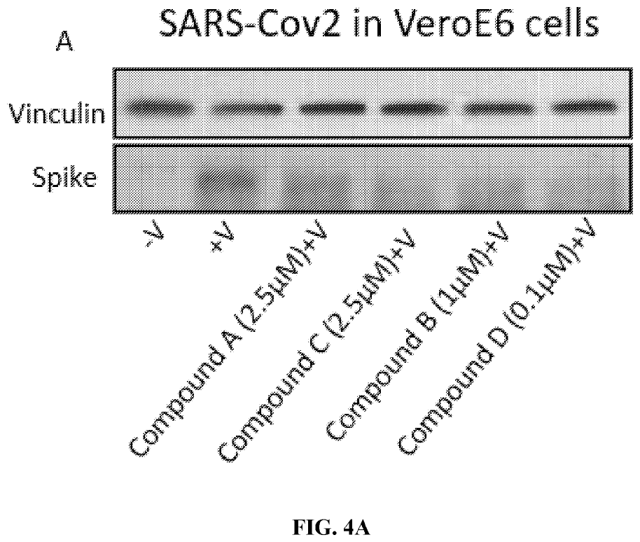

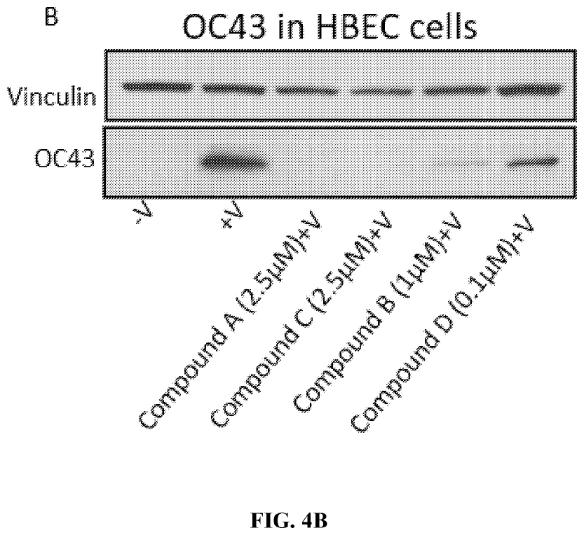

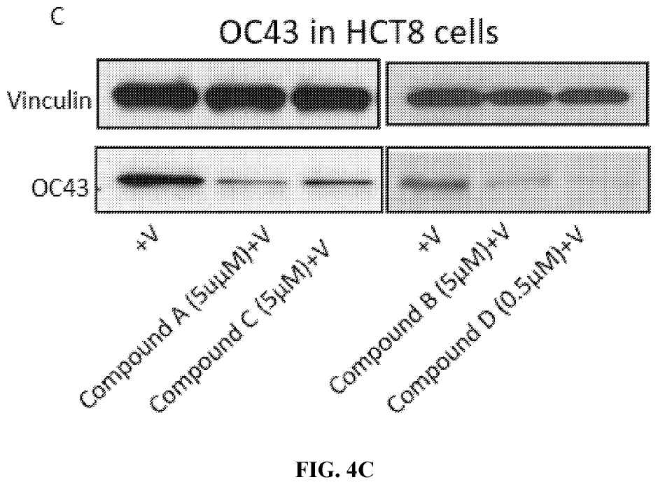

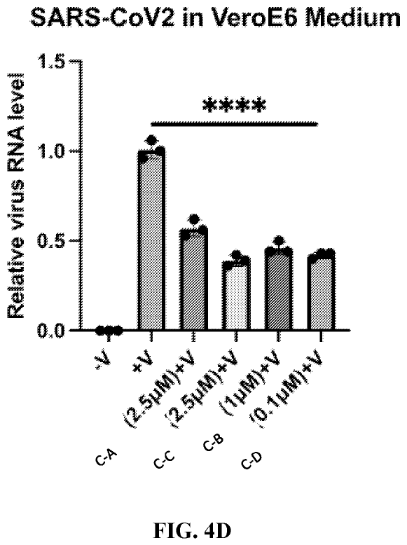

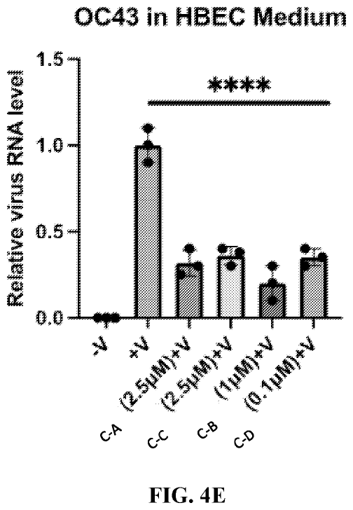

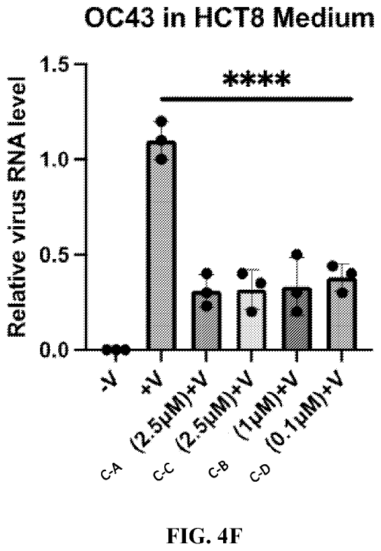

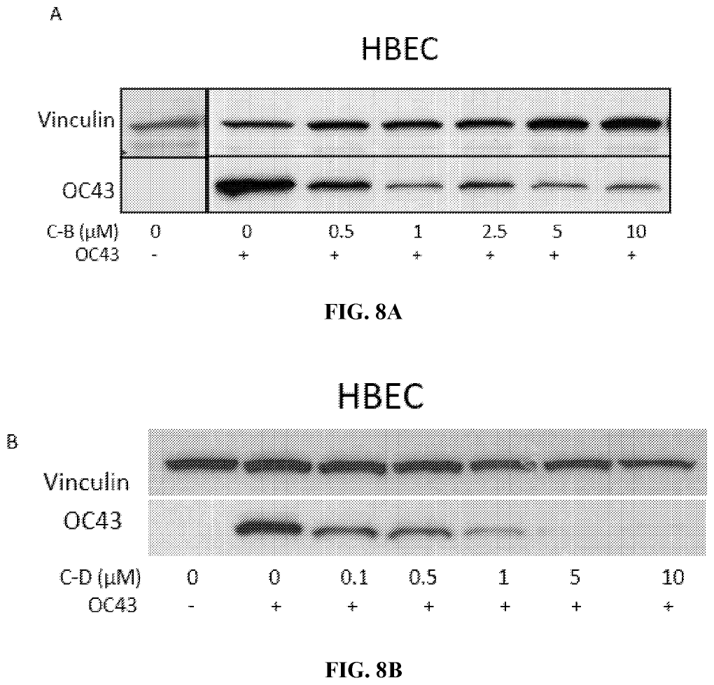

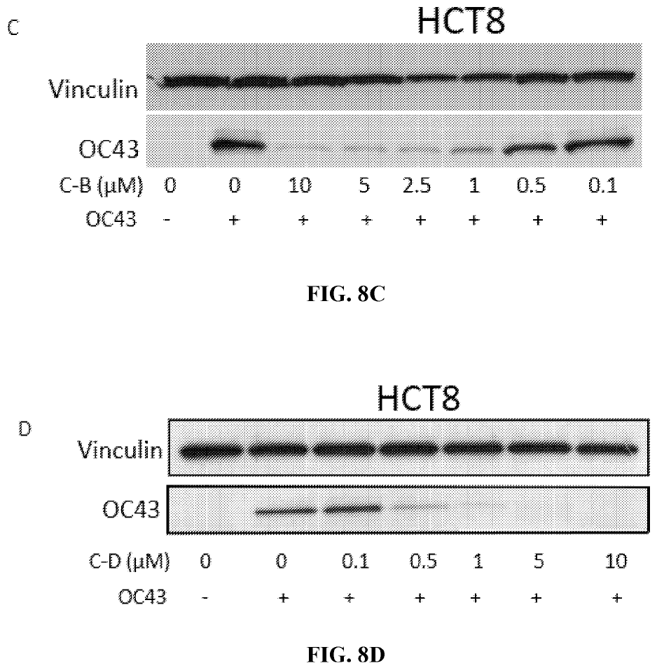

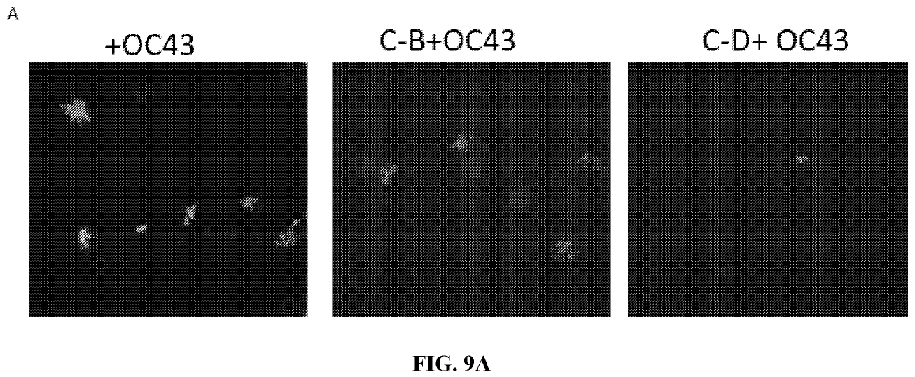

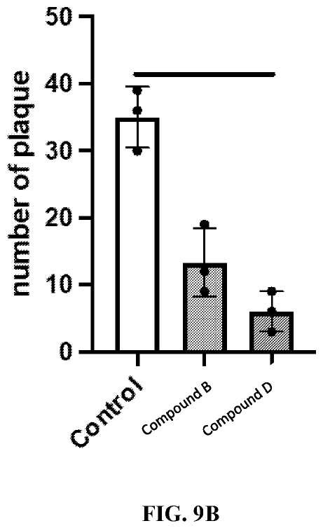

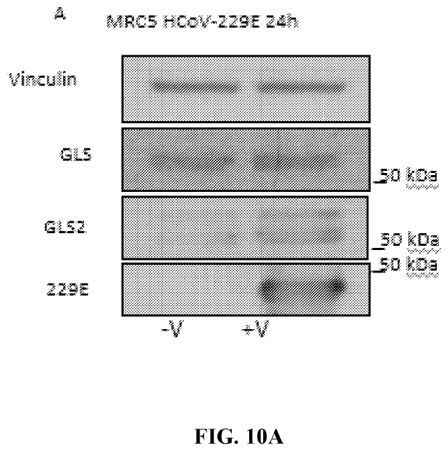

The experiments were started by incubating VeroE6 cells with SARS-CoV-2, and HBECs or HCT8 cells with HCoV-OV43, cultured to 70-80% confluent, either with or without the glutaminase inhibitors, for 3 hours, followed by virus infection (MOI 0.01, 2% FBS RPMI medium) for 1 hour at 330° C. After a 23-hour culture incubation, the cells and their media were collected, and Western blot analyses were performed on the cell lysates to detect the total levels of the SARS-CoV-2 (FIG. 4A) and HCoV-OC43 proteins (FIGS. 4B, and 4C). Virus replication of SARS-CoV2 as read-out by the expression of the viral Spike protein, and HCoV-OC43, determined by the levels of OC43, in the infected cells was significantly suppressed by the glutaminase inhibitors in a dose-dependent manner (FIGS. 4A-C, and FIGS. 8A-8D). These results were confirmed by plaque assays (FIGS. 9A-B) and by qPCR measurements of the virus RNA levels for SARS-CoV2 in the VeroE6 medium (FIG. 4D) and the HCoV-OC43 RNA levels in both HBEC (FIG. 4E) and HCT8 media (FIG. 4F).

### Example 5—Glutaminase Inhibitors do not Block Coronavirus Entry into Host Cells

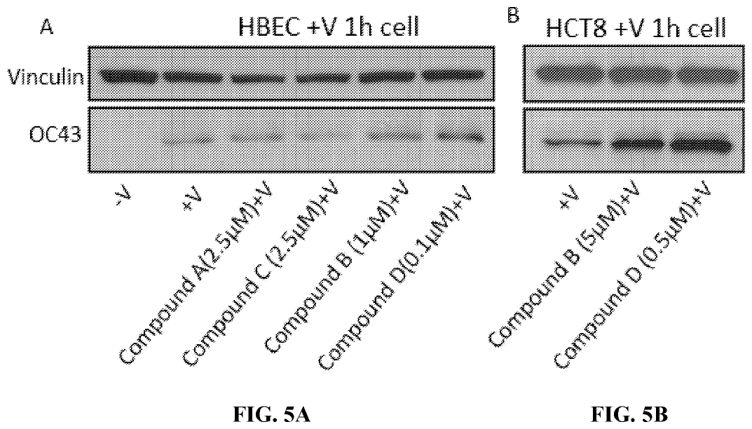

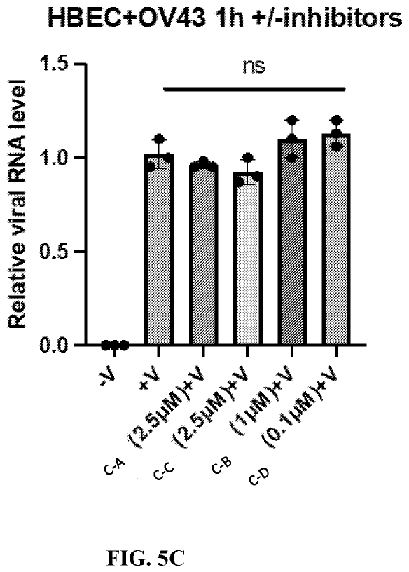

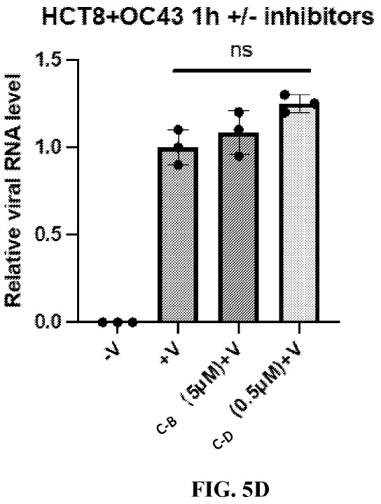

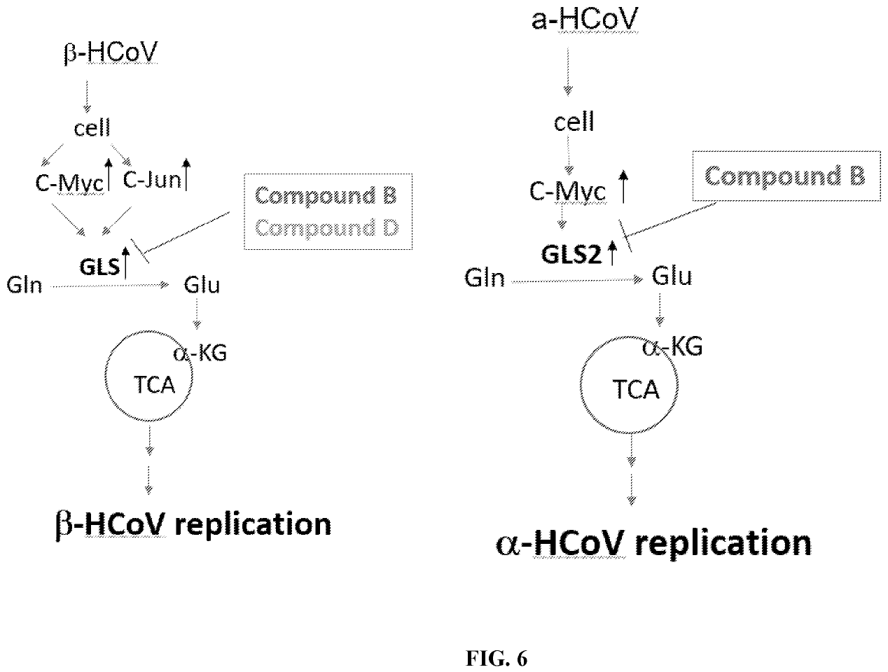

Tests were performed to determine whether GLS inhibitors block Coronaviruses from entering cells. HBECs and HCT8 cells were pretreated with the inhibitors, or were untreated, for 3 hours. The cells were then infected with HCoV-OC43 (MOI 0.01) for 1 hour at 330° C., at which point the cells were collected, and Western Blot analyses were performed to detect the virus levels as read-out by OC43 protein expression (FIGS. 5A and 5B). Viruses were present under all conditions of inhibitor treatments. The presence of viral RNA in the cells as determined by qPCR was also unaffected by inhibitors (FIGS. 5C and 5D). Thus, taken together, these findings indicate that GLS inhibitors block Coronavirus infection not by inhibiting viral entry into host cells but by preventing viral replication.

### Example 6—Coronavirus Infection of MRC5 Lung Epithelial Cells Appears to be Dependent Upon GLS2

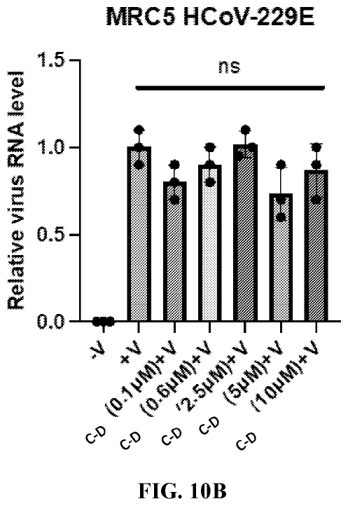

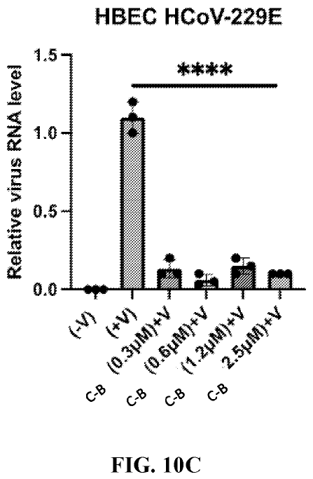

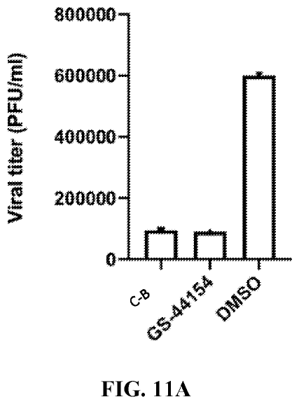

Interestingly, GLS2 expression was upregulated during the alpha sub-group Coronavirus HCoV-229 infection of another lung epithelial host cell, MRC5 (FIG. 10A). GLS expression was greater than GLS2 in MRC5 cells after infecting the cells with HCoC-229E as described above. However, qPCR data showed that the Compound D compound did not significantly block the RNA transcript levels of HCoC-229E detected in the medium indicating that this inhibitor was ineffective in blocking viral infection in MRC5 cells (FIG. 10B). On the other hand, when Compound B compound was examined, which is shown to inhibit GLS2 even more effectively than GLS (Stalnecker et al., “Mechanism by Which a Recently Discovered Allosteric Inhibitor Blocks Glutamine Metabolism in Transformed Cells,” Proc. Natl. Acad. Sci. USA 112(2):394-399 (2014), which is hereby incorporated by reference in its entirety), it was able to block HCoV-229E replication in MRC5 cells (FIG. 0C). Therefore, despite GLS2 being expressed at much lower levels than GLS in MRC5 cells. It nonetheless appears to be essential for Coronavirus infection of these host cells.

### Example 7—Compound B Inhibits Feline Coronavirus FIPV 1 Black Replication

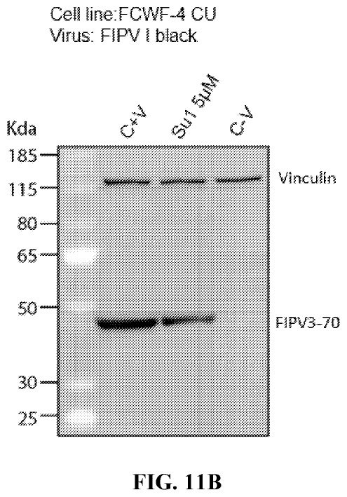

FCWF-4 CU cells (Animal Health Diagnostic Center, Cornell University) were grown in the 75 cm flasks until 80% confluence, and pre-treated the cells with either DMSO, or Compound B (5 uM) for 24 hours. The cells were infected with FIPV 1 Black (MOI 0.01) for 1 hour, and then the cells of each condition were changed to cell growth medium containing either DMSO, Compound B (5 uM) or GS-44154 (it was used as a positive control, it is an anti-Covid-19 drug) for 23 hours. The media containing the viruses were collected. Serial dilutions of each viral medium were made and applied to FCWF-4 CU cells in 12-well plates. 4×10⁵ FCWF-4 CU cells per well were grown on 12-well plates, each dilution was applied and duplicated, and the plates were incubated at 370 C, 5% CO2 for 3 days. The plates were stained with crystal violet (0.1%) after being fixed with 4% PFA, washed with water until the plaques were visualized. The plaques of each condition were counted and the numbers of 10-4 dilution wells were shown and graphed (Table 2, FIG. 11A).

TABLE 2
Concentration and Viral Titer of Plaques of Conditions.

| Condition | Concentration | Viral titer |
|---|---|---|
| DMSO | | 6.5 × 10\hat{ }5 pfu/ml |
| Compound B | 5 μM | 9.5 × 10\hat{ }4 pfu/ml |
| GS | 1 μM | 9 × 10\hat{ }4 pfu/ml |

FCWF-4-CU cells were grown in 75 cm flasks until 80% confluence, pre-treated with DMSO or Compound B (5 μM) for 24 hours, then the cells were infected with FIPV 1 Black (MOI 0.01) for 1 hour, post-treated with DMSO or Compound B in growth medium for 23 hours. The cells were collected from each condition, and a Western blot assay was performed with an anti-feline coronavirus antibody FIPV3-70 (SANTA CRUZ, cat #sc-65653) and an anti-Vinculin antibody as the loading control (SANTA CRUZ, cat #sc-73614). The Western blot assay (FIG. 111B) shows that 1 μM Compound B reduces the production of a feline viral protein and inhibits Feline Coronavirus FIPV 1 Black Replication.

### Example 8—Discussion of Examples 1-7

The glutaminase family of mitochondrial metabolic enzymes has been shown to play important roles in cancer progression due to the ability of its members to catalyze the first step in glutamine metabolism, the hydrolysis of glutamine to glutamate with the accompanying production of ammonia. Altman et al., “From Krebs to Clinic: Glutamine Metabolism to Cancer Therapy,” Nat. Rev. Cancer 16(10):619-634 (2017); De Berardinis and Chandel, “Fundamentals of Cancer Metabolism,” Sci. Adv. 2(5): e1600200 (2016); Greene et al., “STRT5 Stabilizes Mitochondrial Glutaminase and Supports Breast Cancer Tumorigenesis,” Proc. Natl. Acad. Sci. USA 116(52):26625-26632 (2019); Katt et al., “A Tale of Two Glutaminases: Homologous Enzymes With Distinct Roles in Tumorigenesis,” Future Med. Chem. 9(2):223-243 (2017); Lukey et al., “The Oncogenic Transcription Factor c-Jun Regulates Glutaminase Expression and Sensitizes Cells to Glutaminase-targeted Therapy,” Nat. Commun. 7:1-14 (2016); and Best et al., “Glutaminase Inhibition Impairs CD8 T Cell Activation in STK11-/Lkb1-Deficient Lung Cancer,” Cell Metab. 34(6):874-887 (2022), all of which are hereby incorporated by reference in their entirety. By increasing glutaminolysis, cancer cells satisfy their metabolic requirements and glutamine addiction, which are an outcome of the Warburg effect, in which the glycolytic pathway is uncoupled from the TCA cycle. The elevations in glutamine metabolism that occur in cancer cells provide the carbon sources necessary to generate building blocks for biosynthetic processes that underlie their malignant phenotypes. It has been reported that some virus-infected host cells appear to share some of the characteristics of cancer cells and undergo a reprogramming of their metabolism. DeBerardinis et al., “The Biology of Cancer: Metabolic Reprogramming Fuels Cell Growth and Proliferation,” Cell Metab. 7(1):11-20 (2008); Wang et al., “Targeting Mitochondrial Glutaminase Activity Inhibits Oncogenic Transformation,” Cancer Cell 18(3):207-219 (2010); Sanchez et al., “Glycolysis, Glutaminolysis, and Fatty Acid Synthesis Are Required for Distinct Stages of Kaposi's Sarcoma-Associated Herpesvirus Lytic Replication,” J. Virol. 91(10):e02237-16 (2017); and Thai et al., “MYC-induced Reprogramming of Glutamine Catabolism Supports Optimal Virus Replication,” Nat. Commun. 6(1):8873 (2015), all of which are hereby incorporated by reference in their entirety. Here is shown that this is the case for Corona virus infection including Sars-CoV2, by demonstrating that glutamine metabolism in the host cells is essential for their replication.

There are two genes that encode the glutaminase enzymes in mammals and humans, these being GLS and GLS2. GLS is the major form of glutaminase that has been implicated in a variety of human cancers, although GLS2 has also been shown to be important in luminal-subtype breast cancer. Lukey et al., “Liver-Type Glutaminase GLS2 Is a Druggable Metabolic Node in Luminal-Subtype Breast Cancer,” Cell Rep. 29(1):76-88 (2019), which is hereby incorporated by reference in its entirety. In the studies described herein, four different host cell lines and three members of the Coronavirus family have been examined and, in most cases, GLS has been found to be essential for viral replication; however, in one host cell, MRC5, it appears that GLS2 is the glutaminase enzyme required for viral replication. In a wide variety of cancer cells, both GLS expression and its specific activity have been shown to be markedly increased compared to normal healthy cells, whereas in luminal subtype breast cancer cells, GLS2 was upregulated and essential for their growth and survival. Lukey et al., “Liver-Type Glutaminase GLS2 Is a Druggable Metabolic Node in Luminal-Subtype Breast Cancer,” Cell Rep. 29(1):76-88 (2019), which is hereby incorporated by reference in its entirety. The upregulated expression of GLS in cancer cells has been shown to be an outcome of either c-Myc blocking the inhibitory actions of a microRNA (Gao et al., “C-Myc Suppression of miR-23a/b Enhances Mitochondrial Glutaminase Expression and Glutamine Metabolism,” Nature 458(7239):762-765 (2009), which is hereby incorporated by reference in its entirety), or through signaling pathways that result in the activation of the transcription factor c-Jun (Lukey et al., “The Oncogenic Transcription Factor c-Jun Regulates Glutaminase Expression and Sensitizes Cells to Glutaminase-targeted Therapy,” Nat. Commun. 7:1-14 (2016), which is hereby incorporated by reference in its entirety). For host cells infected by Coronaviruses, it is c-Jun activation that upregulates GLS expression. Thus far, very little is known regarding how glutaminase activity is activated either in cancer or virus-infected cells. Both GLS and GLS2 activation requires that the enzyme undergoes a transition from an inactive dimer to a tetramer and then ultimately to a higher-order filament. Jiang et al., “Filamentous GLS1 Promotes ROS-induced Apoptosis Upon Glutamine Deprivation Via Insufficient Asparagine Synthesis,” Mol. Cell 82(10):1821-1835 (2022) and Fang et al., “Filament Formation by Glutaminase Enzymes Drives Catalysis,” bioRxiv. (2023), both of which are hereby incorporated by reference in their entirety. The transition to an activated enzyme requires both the binding of substrate and an anionic activator. Inorganic phosphate is commonly used to serve as an activator in vitro, although the concentrations required (50-100 mM) are unlikely to be achieved in most physiological settings and therefore, it is necessary to determine what type of metabolite or cofactor might serve this function in cells.

There has been a significant amount of effort devoted to developing small molecule inhibitors that target the glutaminase enzymes, given their roles in tumorigenesis. Two of the more common types of GLS inhibitors thus characterized are the Compound A class of molecules and the Compound C family of inhibitory compounds. Wang et al., “Targeting Mitochondrial Glutaminase Activity Inhibits Oncogenic Transformation,” Cancer Cell 18(3):207-219 (2010); Katt et al., “A Tale of Two Glutaminases: Homologous Enzymes With Distinct Roles in Tumorigenesis,” Future Med. Chem. 9(2):223-243 (2017); Lukey et al., “The Oncogenic Transcription Factor c-Jun Regulates Glutaminase Expression and Sensitizes Cells to Glutaminase-targeted Therapy,” Nat. Commun. 7:1-14 (2016); Wicker et al., “Glutaminase Inhibition With Telaglenastat (CB-839) Improves Treatment Response in Combination With Ionizing Radiation in Head and Neck Squamous Cell Carcinoma Models,” Cancer Lett. 502:180-188 (2021)Varghese et al., “The Glutaminase Inhibitor CB-839 (Telaglenastat) Enhances the Antimelanoma Activity of T-cell-mediated Immunotherapies,” Mol. Cancer Ther. 20(3):500-511 (2021); and McDermott et al., “GAC Inhibitors With a 4-hydroxypiperidine Spacer: Requirements for Potency,” Bioorg. Med. Chem. Lett. 29(19):126632 (2019), all of which are hereby incorporated by reference in their entirety. Among the latter are CB839 and our newly developed Compound D, which are significantly more potent than the lead compound Compound C, with CB839 being examined in clinical trials for various cancers. Best et al., “Glutaminase Inhibition Impairs CD8 T Cell Activation in STK11-/Lkb1-Deficient Lung Cancer,” Cell Metab. 34(6):874-887 (2022); Harding et al., “A Phase I Dose-escalation and Expansion Study of Telaglenastat in Patients With Advanced or Metastatic Solid Tumors,” Clin. Cancer Res. 27(18):4994-5003 (2021); and Riess et al., “Phase 1 Trial of MLN0128 (Sapanisertib) and CB-839 HCl (Telaglenastat) in Patients With Advanced NSCLC (NCI 10327): Rationale and Study Design,” Clin. Lung Cancer 22(1):67-70 (2021), all of which are hereby incorporated by reference in their entirety. The Compound C series of compounds bind within the interface where two GLS dimers come together to form a tetramer and stabilize an inactive tetrameric species that is incapable of forming higher order filament-like structures. The Compound A class of GLS inhibitors which includes the lead compound, Compound A, and our more potent analog Compound B function in a distinct manner from the Compound C class of compounds, by preferentially binding initially to GLS monomers and then stabilizing inactive dimers. Both classes of allosteric GLS inhibitors effectively blocked Coronavirus replication in HBECs, HCT8 and VeroE6 cells, as read out by the synthesis of a viral coat protein and when assaying viral RNA transcript levels, matching the effects observed when knocking down GLS expression in host cells. However, in MRC5 cells, only the Compound A class of compounds and specifically Compound B was effective at inhibiting viral replication because of the required role of GLS2, which is relatively insensitive to the Compound C-family of compounds.

Current anti-Coronaviruses drugs target proteins essential for virus replication, including RNA polymerases (Seifert et al., “Inhibition of sars-cov-2 Polymerase by Nucleotide Analogs From a Single-molecule Perspective,” Elife 10:e70968 (2021); Mouffouk et al., “Flavonols as Potential Antiviral Drugs Targeting SARS-CoV-2 Proteases (3CLpro and PLpro), Spike Protein, RNA-dependent RNA Polymerase (RdRp) and Angiotensin-converting Enzyme II Receptor (ACE2),” Eur. J. Pharmacol. 891:173759 (2021); and Tian et al., “RNA-dependent RNA Polymerase (RdRp) Inhibitors: The Current Landscape and Repurposing for the COVID-19 Pandemic,” Eur. J. Med. Chem. 213:113201 (2021), all of which are hereby incorporated by reference in their entirety), the Coronavirus spike protein receptor, ACE2 (Hoffmann et al., “SARS-CoV-2 Cell Entry Depends on ACE2 and TMPRSS2 and Is Blocked by a Clinically Proven Protease Inhibitor,” Cell 181(2):271-280 (2020), which is hereby incorporated by reference in its entirety), and the serine protease, TMPRSS2 (Hoffmann et al., “SARS-CoV-2 Cell Entry Depends on ACE2 and TMPRSS2 and Is Blocked by a Clinically Proven Protease Inhibitor,” Cell 181(2):271-280 (2020), which is hereby incorporated by reference in its entirety, which is necessary to promote high affinity interactions between spike proteins and their host cell receptors. the work described herein has focused on blocking the metabolic activities required to generate building blocks for biosynthetic processes and the energy supply necessary for Coronavirus replication. Because the glutaminase enzymes are often much more highly expressed and activated in cancer (De Berardinis and Chandel, “Fundamentals of Cancer Metabolism,” Sci. Adv. 2(5): e1600200 (2016); Vander Heiden and DeBerardinis, “Understanding the Intersections Between Metabolism and Cancer Biology.” Cell 168(4):657-669 (2017); Pavlova and Thompson, “The Emerging Hallmarks of Cancer Metabolism,” Cell Metab. 23(1):27-47 (2016), all of which are hereby incorporated by reference in their entirety) and viral infected host cells (Mayer et al., “Hijacking the Supplies: Metabolism as a Novel Facet of Virus-host Interaction,” Front Immunol. 10(JULY):1-12 (2019); Thai et al., “MYC-induced Reprogramming of Glutamine Catabolism Supports Optimal Virus Replication,” Nat. Commun. 6(1):8873 (2015); Fontaine et al., “Vaccinia Virus Requires Glutamine but Not Glucose for Efficient Replication,” J Virol. 88(8):4366-4374 (2014); and Bharadwaj et al., “SARS-CoV-2 and Glutamine: SARS-CoV-2 Triggered Pathogenesis via Metabolic Reprograming of Glutamine in Host Cells,” Front. Mol. Biosci. 7(January):1-14 (2021), all of which are hereby incorporated by reference in their entirety), compared to normal healthy cells, small molecule inhibitors targeting these enzymes offer a potentially safe therapeutic strategy. There have been reports suggesting that the replication of various viruses is dependent upon glutamine metabolism (Thai et al., “MYC-induced Reprogramming of Glutamine Catabolism Supports Optimal Virus Replication,” Nat. Commun. 6(1):8873 (2015) and Fontaine et al., “Vaccinia Virus Requires Glutamine but Not Glucose for Efficient Replication,” J Virol. 88(8):4366-4374 (2014), which are hereby incorporated by reference in their entirety). Herein is shown that three Coronaviruses including SARS-CoV2 require glutamine metabolism for their replication and are susceptible to inhibition by allosteric glutaminase inhibitors. Some newly designed glutaminase inhibitors are especially effective at blocking Coronavirus infection and may serve as new lead compounds toward the design of even more potent and novel anti-viral drug candidates.

Finally, these findings raise a number of interesting questions and lines of investigation for future studies. They include determining how virus infection triggers the necessary signals to upregulate glutaminase expression and elucidating the mechanism responsible for the activation of GLS catalytic activity. It also will be of interest to establish whether glutaminase filament-like structures form within virus infected host cells, similar to cancer cells (Feng et al., “Formation of Oligomeric Filament-like Structures of Glutaminase is Directly Coupled to Catalytic Activity,” Deposited in bioRxiv (2023) and Ferreira et al., “Active Glutaminase C Self-assembles Into a Supertetrameric Oligomer That Can be Disrupted by an Allosteric Inhibitor,” J. Biol. Chem. 288:28009-28020 (2013), both of which are hereby incorporated by reference in their entirety). If so, do these higher order oligomeric structures serve as a scaffold for a metabolic complex necessary for satisfying the requirements for viral replication, and can strategies be designed specifically block their formation and thus yield additional classes of anti-viral therapeutics.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of treating a virus infection in a subject, the method comprising:
administering to the subject a compound of either:
(A) formula (I) 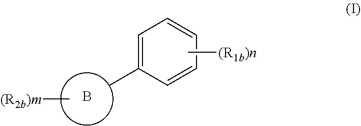
wherein:
m and n are integers from 1 to 4;
B is a substituted or unsubstituted mono or polycycle, wherein the monocycle is an aryl ring, a heteroaryl ring, a heterocyclic ring or a cycloalkyl ring and wherein the polycycle comprises any combination of one or more aryl rings, one or more heteroaryl rings, one or more heterocyclic rings, or one or more cycloalkyl rings, with each heterocyclic ring containing from 1 to 5 heteroatoms selected from the group consisting of nitrogen, sulfur, and oxygen;
R_{1b} and R_{2b} are independently H, oxo, OH, OR_{3b}, halogen, CN, NO₂, COOH, NH₂, NHR_{3b}, NR_{3b}R_{4b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; and
R_{3b} and R_{4b} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl containing from 1 to 5 heteroatoms selected from the group consisting of nitrogen, sulfur, and oxygen,
or
(B) formula (II) 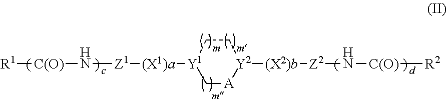
wherein A is —CH₂— or -Q-(CH₂)ₙ—;
each ----- is optionally present, and when present is a single or double bond;
Y¹ and Y² are each independently N or C with the proper valency;
Q is —S—, —O—, or —CH₂—;
X¹ and X² are each independently —NH—, —O—, —CH₂—O—, —NH—CH₂—, or —N(CH₃)—CH₂—;
a and b are each independently 0 or 1;
c and d are each independently 0 or 1;
m, m′, m″, and n are each independently an integer of 0-2;
Z¹ and Z² are each independently a heterocyclic; and
R¹ and R² are each independently optionally substituted alkyl, optionally substituted aralkyl, optionally substituted cycloalkyl, amino, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl;
provided that if c is 0 and d is 0, then R¹ and R² are both amino;
provided that if c is 1 and d is 1, then both R¹ and R² are not amino;
provided that if c is 0 and d is 1, then R¹ is amino and R² is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl; and
provided that if c is 1 and d is 0, then R² is amino and R¹ is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl,
or a pharmaceutically acceptable salt of formula (I) or formula (II).

2. The method of claim 1, wherein the compound is a compound of formula (I).

3. The method of claim 2, wherein formula (I) comprises formula (Ia): 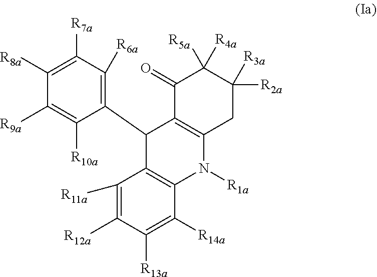
wherein:
R₁ₐ is independently H, OH, OR₁₅ₐ, C₁-C₆ alkyl, C₂-C₆ alkenyl, C₂-C₆ alkynyl, R₁₅ₐC(O)—, R₁₅ₐOC(O)—, R₁₅ₐS(O)—, or R₁₅ₐS(O)₂;
R₂ₐ, R₃ₐ, R₄ₐ, and R₅ₐ are each independently H, OH, NH₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the aryl, heteroaryl, and aryl C₁-C₆ alkyl, are optionally substituted from 1 to 3 times with substituents independently selected from the group consisting of halogen, OH, NH₂, C₁-C₆ alkyl, C₂-C₆ alkenyl, C₁-C₆ alkoxy, SH, and C₁-C₆ thioalkyl;
R₆ₐ, R₇ₐ, R₈ₐ, R₉ₐ, and R₁₀ₐ are each independently H, halogen, NO₂, OH, OR₁₅ₐ, SR₁₅ₐ, NH₂, NHR₁₅ₐ, NR₁₅ₐR₁₆ₐ, R₁₅ₐC(O)—, R₁₅ₐOC(O)—, R₁₅ₐC(O)O—, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; or
R₆ₐ and R₇ₐ, R₇ₐ and R₈ₐ, R₈ₐ and R₉ₐ, or R₉ₐ and R₁₀ₐ can combine to form a heterocyclic ring; and
R₁₁ₐ, R₁₂ₐ, R₁₃ₐ, R₁₄ₐ, R₁₅ₐ, and R₁₆ₐ are each independently H, halogen, OH, NO₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, each one of R₁₁ₐ-R₁₆ₐ optionally independently substituted with NH₂, OH, halogen, COOH, NO₂, and CN; or R₁₁ₐ and R₁₂ₐ, R₁₂ₐ and R₁₃ₐ, or R₁₃ₐ and R₁₄ₐ can combine to form an optionally substituted aromatic ring.

4. The method of claim 2, wherein the compound of formula (I) is selected from the group consisting of:

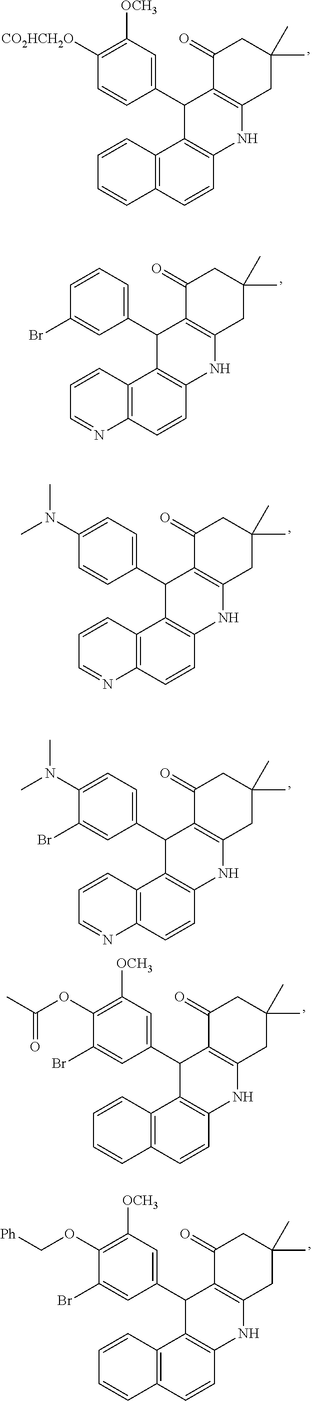

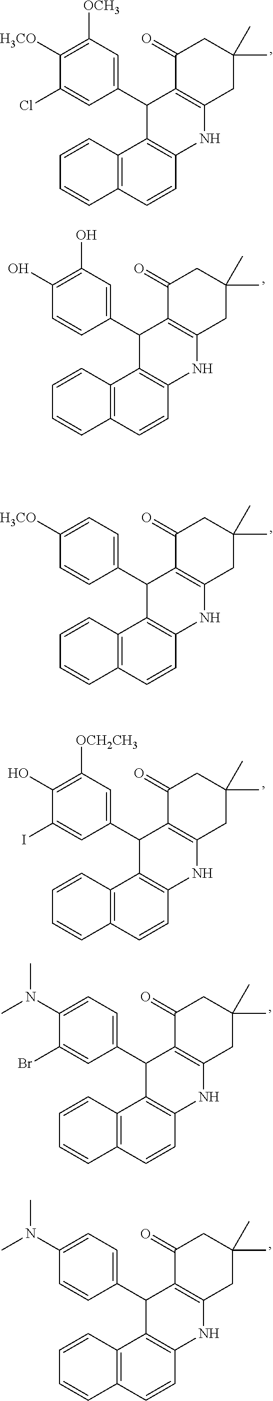

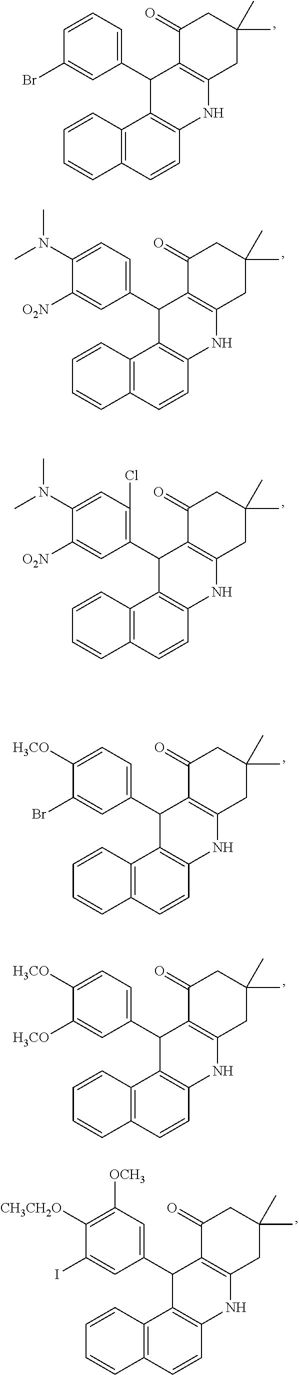

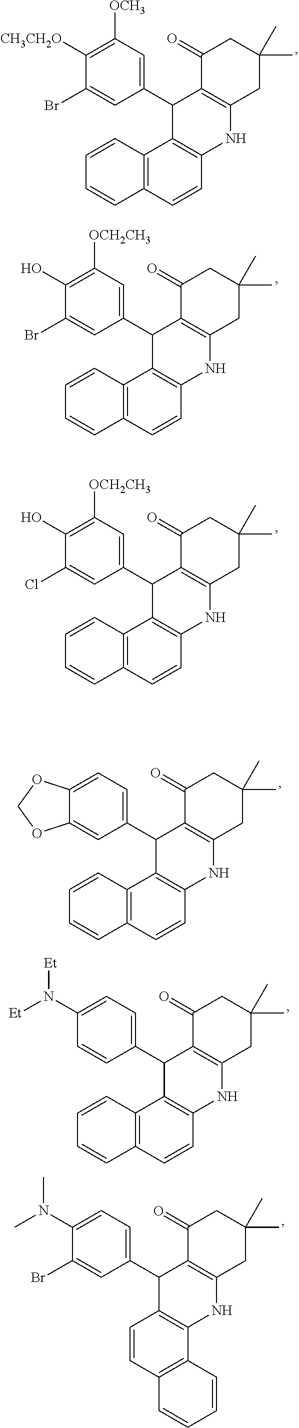

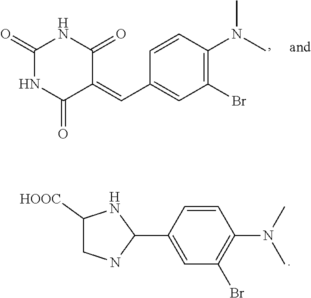 [SMILES: CN(C)c1ccc(C=C2C(=O)NC(=O)NC2=O)cc1Br ; CN(C)c1ccc(C2NCC(C(=O)O)N2)cc1Br]

5. The method of claim 2, wherein the compound of formula (I) is:

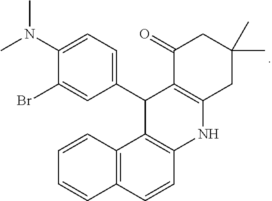

6. The method of claim 2, wherein the compound of formula (I) is:

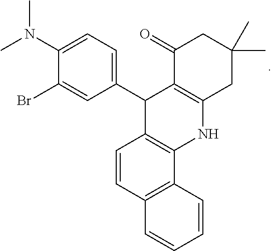

7. The method of claim 1, wherein the compound is a compound of formula (II).

8. The method of claim 7, wherein A is —S—(CH₂)ₙ—.

9. The method of claim 7, wherein formula (II) comprises formula (IIa) 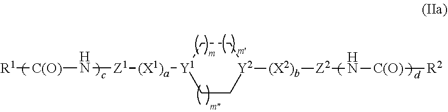
wherein
X¹ and X² are each independently —NH—, —O—, —CH₂—O—, —NH—CH₂—, or —N(CH₃)—CH₂—, provided that when at least one of X¹ and X² is —CH₂—O—, —NH—CH₂—, or —N(CH₃)—CH₂—, then the —CH₂— is directly connected to 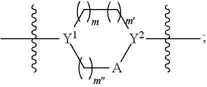
provided that if Y¹ and Y² are each C, then a is 1 and b is 1;
provided that if Y¹ and Y² are each N, then a is 0 and b is 0;
provided that if Y¹ is N and Y² is C, then a is 0 and b is 1; and
provided that if Y¹ is C and Y² is N, then a is 1 and b is 0.

10. The method of claim 9, wherein 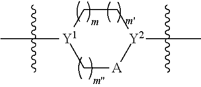
m is a 3, 4, 5, 6 or 7 membered N-heterocycle.

11. The method of claim 9, wherein 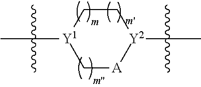
is piperidinyl.

12. The method of claim 9, wherein 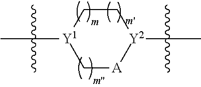
is selected from the group consisting of:

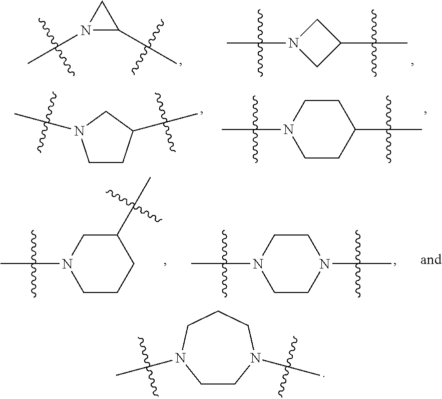

13. The method of claim 9, wherein 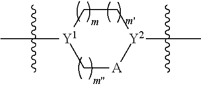
is a 3, 4, 5, 6 or 7 membered cycloalkylene where Y¹ and Y² are carbon.

14. The method of claim 11, wherein 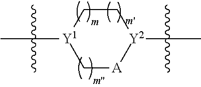
is selected from the group consisting of:

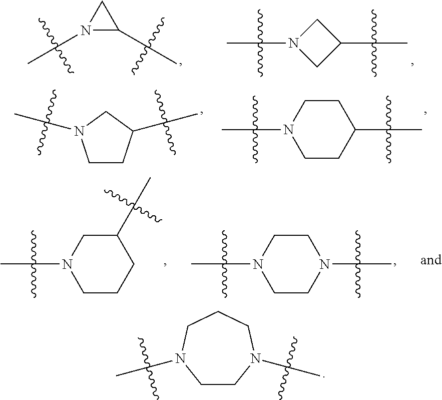

15. The method of claim 7, wherein the compound is selected from the group consisting of:

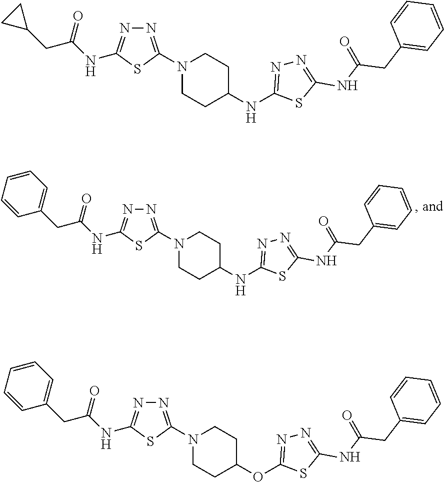 [SMILES: O=C(Cc1ccccc1)Nc1nnc(NC2CCN(c3nnc(NC(=O)CC4CC4)s3)CC2)s1 ; O=C(Cc1ccccc1)Nc1nnc(NC2CCN(c3nnc(NC(=O)Cc4ccccc4)s3)CC2)s1 ; O=C(Cc1ccccc1)Nc1nnc(OC2CCN(c3nnc(NC(=O)Cc4ccccc4)s3)CC2)s1]

16. The method of claim 1, wherein Z¹ and Z² of formula (I) or formula (II) are independently selected from the group consisting of:

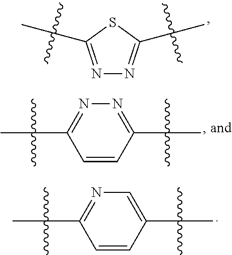

17. The method of claim 1, wherein the compound of formula (II) is:

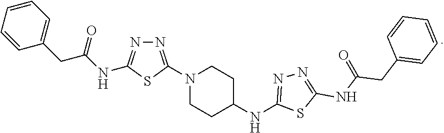 [SMILES: O=C(Cc1ccccc1)Nc1nnc(NC2CCN(c3nnc(NC(=O)Cc4ccccc4)s3)CC2)s1]

18. The method of claim 1, wherein the compound of formula (II)

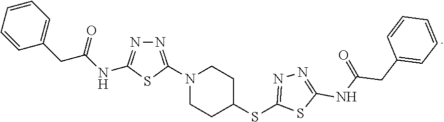 [SMILES: O=C(Cc1ccccc1)Nc1nnc(SC2CCN(c3nnc(NC(=O)Cc4ccccc4)s3)CC2)s1]

19. The method of claim 1, wherein the virus infection is a coronavirus infection.

20. The method of claim 19, wherein the virus infection is a severe acute respiratory syndrome coronavirus 2 (SARS-CoV-2), severe acute respiratory syndrome coronavirus (SARS-CoV), Middle East respiratory syndrome-related coronavirus (MERS-CoV), Human coronavirus HKU1 (HCoV-HKU1), Human coronavirus OC43 (HCoV-OC43), Human coronavirus NL63 (HCoV-NL63), or Human coronavirus 229E (HCoV-229E) infection.

21. The method of claim 20, wherein the virus infection is a SARS-CoV2 infection.

22. The method of claim 1 further comprising:
selecting a subject with a virus infection.

23. The method of claim 22, wherein the selected subject has SARS, MERS, or COVID-19.

24. The method of claim 23, wherein the selected subject has mild, moderate, or severe symptoms or exhibits no symptoms associated with SARS, MERS, or COVID-19.

25. The method of claim 1, wherein the virus infection is a feline coronavirus.

26. The method of claim 25, wherein the feline coronavirus is feline enteric coronavirus (FECV) or feline infectious peritonitis virus (FIPV).

27. The method of claim 1 further comprising:
administering one or more additional therapeutic agent in conjunction with said administering the compound of formula (I) or the compound of formula (II).

28. The method of claim 27, wherein the additional therapeutic agent is an antiviral compound, an immune modulator, a convalescent plasma, or a combination thereof.

29. The method of claim 1, wherein said administering is carried out orally, by intravenous injection, by inhalation, by intranasal instillation, topically, transdermally, parenterally, subcutaneously, by intra-arterial injection, by intramuscular injection, intraplurally, intraperitoneally, or by application to mucous membrane.

30. The method of claim 1 further comprising:
repeating said administering.

31. The method of claim 1, wherein the subject is a mammal.

32. The method of claim 31, wherein the subject is a human.

33. The method of claim 31, wherein the subject is a feline.

34. The method of claim 1, wherein the subject is an infant or a juvenile.

35. The method of claim 1, wherein the subject is an adult.

36. The method of claim 1, wherein said treating further comprises treating a condition resulting from a virus infection.

37. The method of claim 1, wherein the compound is administered in a concentration range of between about 0.01 mg/kg to about 25 mg/kg.

38. A method of inhibiting viral replication, said method comprising:
contacting one or more cells infected with a virus with an effective amount of a compound of either:
(A) formula (I) 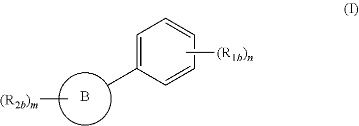
wherein:
m and n are integers from 1 to 4;
B is a substituted or unsubstituted mono or polycycle, wherein the monocycle is an aryl ring, a heteroaryl ring, a heterocyclic ring or a cycloalkyl ring and wherein the polycycle comprises any combination of one or more aryl rings, one or more heteroaryl rings, one or more heterocyclic rings, or one or more cycloalkyl rings, with each heterocyclic ring containing from 1 to 5 heteroatoms selected from the group consisting of nitrogen, sulfur, and oxygen;
R_{1b} and R_{2b} are independently H, oxo, OH, OR_{3b}, halogen, CN, NO₂, COOH, NH₂, NHR_{3b}, NR_{3b}R_{4b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; and
R_{3b} and R_{4b} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl containing from 1 to 5 heteroatoms selected from the group consisting of nitrogen, sulfur, and oxygen,
or
(B) formula (II) 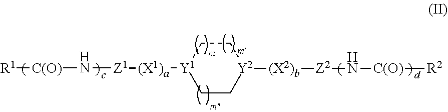
wherein A is —CH₂— or -Q-(CH₂)ₙ—;
each ----- is optionally present, and when present is a single or double bond;
Y¹ and Y² are each independently N or C with the proper valency;
Q is —S—, —O—, or —CH₂—;
X¹ and X² are each independently —NH—, —O—, —CH₂—O—, —NH—CH₂—, or —N(CH₃)—CH₂—;
a and b are each independently 0 or 1;
c and d are each independently 0 or 1;
m, m′, m″, and n are each independently an integer of 0-2;
Z¹ and Z² are each independently a heterocyclic; and
R¹ and R² are each independently optionally substituted alkyl, optionally substituted aralkyl, optionally substituted cycloalkyl, amino, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl;
provided that if c is 0 and d is 0, then R¹ and R² are both amino;
provided that if c is 1 and d is 1, then both R¹ and R² are not amino;
provided that if c is 0 and d is 1, then R¹ is amino and R² is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl; and
provided that if c is 1 and d is 0, then R² is amino and R¹ is optionally substituted alkyl, optionally substituted aralkyl, optionally substituted cycloalkyl, optionally substituted heteroaralkyl, optionally substituted alkylalkoxy, optionally substituted alkylaryloxy, optionally substituted aryl, optionally substituted heteroaryl, or optionally substituted heterocycloalkyl with substituents independently selected from the group consisting of halogen, OH, NH₂, C₁-C₆ alkyl, C₂-C₆ alkenyl, C₁-C₆ alkoxy, SH, and C₁-C₆ thioalkyl,
or a pharmaceutically acceptable salt of formula (I) or formula (II).

39-61. (canceled)
